(19) United States
(12) Patent Application Publication
(10) Pub. No.: US 2010/0040601 A1
(43) Pub. Date: Feb. 18, 2010

(54) COMPOSITIONS AND METHODS FOR TREATING HERPES SIMPLEX VIRUS INFECTIONS AND RELATED DISEASES

(76) Inventors: Edouard M. Cantin, Alhambra, CA (US); Ramakrishna Chandran, Duarte, CA (US); Patric Lundberg, Carrollton, VA (US)

Correspondence Address: PERKINS COIE LLP, POST OFFICE BOX 1208, SEATTLE, WA, 98111-1208, US

(21) Appl. No.: 12/482,418

(22) Filed: Jun. 10, 2009

Related U.S. Application Data
(60) Provisional application No. 61/060,339, filed on Jun. 10, 2008.

(51) Int. Cl. A61K 39/395; C07K 16/00; A61P 31/22

(52) U.S. Cl. 424/130.1; 530/387.1; 530/389.1

(57) ABSTRACT

A method of treatment or prophylaxis of herpes infection and associated diseases by administration of compositions comprising pooled immunoglobulins. The active component of IVIG includes sialylated IgG species. Treatment and prophylaxis of herpes infection include herpes simplex virus 1 infection, its associated encephalitis and herpes stromal keratitis.

### PRIORITY CLAIM

This application claims the benefit of U.S. Provisional Application Ser. No. 61/060,339, filed Jun. 10, 2008, which is incorporated herein by reference in its entirety.

### GOVERNMENT INTEREST

This invention was made with Government support of Grant number EY-013814 awarded by the National Institutes of Health. The government has certain rights in this invention.

### FIELD OF THE INVENTION

The present invention relates generally to diseases caused by herpes infection and, more specifically, to use of compositions comprising pooled immunoglobulin for the treatment and/or prophylaxis of herpes infection and its associated disease states.

### BACKGROUND

Herpes simplex is a disease caused by Herpes simplex viruses (HSV). Infection with the virus is categorized according to certain distinct disorders based upon the site of infection. Herpes encephalitis is an acute inflammation of the brain occurring in about two thousand individuals each year in the Untied States. Symptoms include fever, headache and photophobia for several days, followed by behavior changes, seizures, hemiparesis and depressed level of consciousness. Herpes simplex virus type 1 is the major cause of sporadic encephalitis, which despite treatment with antiviral drugs is still associated with high mortality (˜20%) and serious neurological sequelae in many survivors. Adult HSV encephalitis can reflect primary infection, reinfection, or reactivation of latent virus. Death due to fatal HSV encephalitis (HSE) is commonly ascribed to damage resulting from HSV replication in the CNS, though recent clinical experience and studies in experimental animal models have implicated pathogenic immune responses. Treatment is usually symptomatic. There are not many effective specific antiviral agents and those that have been reliably tested are available only for a few viral agents. The most common treatment currently available for HSE is with the intravenous infusion of the antiviral agent, acyclovir. However, this treatment can cause undesired side effects, such as renal insufficiency, and relapse is common.

A defining characteristic of herpes viruses is their ability to establish life-long latent infections in the natural host and to reactivate periodically facilitating widespread dissemination in the population. HSV-1 and HSV-2 are closely related large DNA viruses that establish enduring latent infections in the host accounting for their widespread prevalence among humans. They are comparably pathogenic and cause similar diseases that depend on the route of infection, virus strain, host immunological status and unidentified host genetic determinants (109, 131).

Reactivated rather than primary HSV infections are associated with serious diseases including herpes keratitus (HSK), a leading cause of blindness in developed countries and herpes simplex encephalitis (HSE), which though less common remains a debilitating disease associated with high mortality and morbidity. Primary HSV infections are usually mild or asymptomatic and despite vigorous host immune responses that rapidly eradicate replicating virus, latent infection of sensory ganglionic neurons is inevitable. Initially, HSV replicates in epithelial tissues and then invades sensory neurons by which it spreads to the corresponding sensory ganglia and CNS. Details of HSV pathogenesis have been elucidated primarily by studies using small animal models like the mouse and guinea pig that recapitulate aspects of human HSV infection to various extents (45, 108, 120). An important concept is that the outcome of HSV infection is a function of complex host-virus interactions. Damage to the host is not determined solely by HSV strain virulence nor does it result exclusively from weak host defenses. Rather, damage due to virus cytopathology can occur when the host response fails to control the virus and at the other extreme, when an exaggerated immune response damages the host, which may favor viral spread and replication (14, 15).

HSV is the leading cause of sporadic non-endemic encephalitis accounting for as many as 20% of acute viral encephalitis cases, with an associated mortality rate of 70% when untreated; in the absence of treatment prognosis is poor with only 2.5% of patients regaining normal function (36, 56). While treatment with ACV improves these statistics, about 20% of patients still suffer serious clinical complication, even death. Additionally, infants infected with HSV may be at even greater risk because of disseminated disease or encephalitis that is often fatal or results in serious neurological and cognitive complications in survivors. When promptly treated, 10% of children still do not survive the infection, and learning and other mental debilities occur in survivors (16, 124, 129). Importantly, the mechanisms underlying developments of severe HSE remain undefined. It has long been suspected, based on clinical observations and animal studies, that HSE and other HSV diseases result from immune pathology and consequently might be exacerbated by treatments designed to boost antiviral immunity (1, 2, 110).

MRI observations of HSE in patients and experimental animal models have revealed evidence of persistent inflammatory activity and chronic progressive tissue damage months to years from onset of symptoms despite early treatment with ACV, the recommended antiviral drug (30, 76, 77, 128). Such long-term MRI abnormalities are commonly associated with high morbidity and mortality (78). Increasingly, there are reports of HSE worsening despite ACV treatment (41, 87), and in some cases marked improvement was seen following treatment with combined immunosuppressant plus antiviral drug (46, 101), which implicates immune mechanisms in HSE. Persistent neuroinflammation visualized by detection of activated microglia/macrophages at sites remote from focal HSE lesions using [11C] ®-PK11195-PET was recently documented for two patients (9). The correlation of long-term functional deficits with significant PET signal increases (in the affected limbic system) reinforces the perception that the long-term immune activation, frequently observed in HSE in the absence of viral replication, is detrimental (61). In a recent study of experimental HSE, combined treatment of mice with ACV and corticosteroids significantly reduced long-term MRI abnormalities whereas ACV treatment alone did not (79). Notably, corticosteroids did not affect HSV titers in the CNS; titers were comparable in both groups of ACV treated mice and significantly lower than untreated mice (79, 122). Results from experimental studies and those of others considered in light of clinical observations makes a compelling case for immune mechanisms, especially those involving innate cells, being causally involved in the development of HSE. Clearly, uncovering the pathogenic immune mechanisms underlying development of HSE is critical for developing new therapies targeting both the virus and specific immune effector pathways.

The immune mechanisms responsible for control of HSV infections, particularly CNS infections are complex and ill defined. The fact that natural immunity provides only partial protection from recurrent and secondary infections, the lack of an effective vaccine and the emergence of drug resistant HSV strains in immunosupressed patients prompted early exploration of antibody therapy for HSV infection (21, 29, 130).

Many human pathogens including herpes viruses encode IgG Fc receptors (FcgRs). The HSV gE and gi glycoproteins play a vital role in cell-to-cell spread and they also form a hetero-oligomeric complex that constitutes the HSV IgG FcgR. gE alone acts as a low affinity FcgR binding IgG aggregates but not IgG monomers, while the gE-gI complex acts as a high affinity FcgR binding both IgG monomers and aggregates (5, 25). The HSV FcgR preferentially binds anti-HSV IgG by a process called ‘antibody bipolar bridging’, which occurs when the Fab domain binds its antigenic target and the Fc domain of the same molecule binds the viral FcgR that is expressed on the same cell (8, 32).

Sustained immunomodulatory activity of IVIG, namely induction of an IFN-g refractory state in human and mouse macrophages was recently reported (96). Macrophages exposed to IVIG for >10 hr failed to phosphorylate STAT1 in response to IFN-g and consequently STAT-1 target genes were not induced. The IVIG inhibitory effect was dependent on FcgRIII expression but not FcgRIIb expression, which reveals an unexpected inhibitory role for the activating FcgRIII in mediating suppression of IFN-g signaling. This finding parallels a recent report that FcaR1 behaves as a bifunctional activating/inhibitory receptor highlighting the finely tuned mechanisms that have evolved to regulate inflammation (97). Inhibition of macrophage responses to IFN-g can contribute to the anti-inflammatory properties of IVIG (95).

Given the complexity and variability of the virus and its effect of animals of various genetics, there is a need for a more effective and reliable treatment of HSV. The present compositions, methods, and animal models for further testing satisfy this need and provide related advantages.

### SUMMARY

Pooled human Immunoglobulin (IVIG) consists of the polyclonal IgG fraction pooled from thousands of donors. Consequently, polyreactive natural antibodies and antibodies specific for allotypic antigens must be represented in the pool. IVIG therapeutic actions involve several discrete, kinetically distinct, mechanisms of action evident in specific disease states. IVIG protection, as shown in the novel models discussed herein, provides new mechanistic insights because the virus expresses immune evasion molecules including a decoy FcR. The IgG Fc domain is required but insufficient for protection of susceptible 129 mice from fatal HSE. The potential for IVIG to suppress not only antibody-mediated autoimmune disease but also chronic or acute inflammatory states in which damage is caused by activated leukocytes is disclosed in the present invention.

IVIG mechanisms have been elucidated herein by studying HSV infection of B6- and 129-Rag/E strains involved in suppressing inflammation and virus replication (FIG. 15). A single IVIG treatment of the present disclosure is able to prevent fatal HSE developing in 129-E that lack mast cells but not 129-Rag mice that have mast cells. The dependence of IVIG protection on the Fc domain of the IgG molecule is unexpected since this dependence applies also to mouse IgG antibodies whose Fc domain is not bound by the HSV FcgR.

Accordingly, in one aspect, the invention relates to a method of treatment or prophylaxis of herpes simplex virus (HSV) infections in mammals by administering intravenously to the mammal an effective amount of a composition for intravenous injection of pooled human immunoglobulin or recombinantly produced mono-oligo- or polyclonal immunoglobulins or immunoglobulin domains (IVIG). The mammal may be a neonatal, infant, child, or adult human. The mammal can be immunocompromised and/or suffer from a herpetic disease. The herpetic disease can be encephalitis, pneumonia, hepatitis, herpes ocularis, chickenpox, shingles, zoster oticus, zoster varicellosus, keratitis, herpes digitalis, herpes facialis, herpes genitalis, herpes gladiatorum, or herpes stomatitis. More than one of any of the foregoing can characterize the herpetic disease afflicting the mammal.

In another aspect, the invention relates to a method of treatment or prophylaxis of other CNS viral and non-viral inflammatory diseases in mammals by administering intravenously to the mammal an effective amount of a composition for intravenous injection of pooled human immunoglobulin (IVIG). The mammal can be a neonatal, infant, child, or adult human. The mammal can be immunocompromised and/or suffer from a herpetic disease. The herpetic disease can be encephalitis, pneumonia, hepatitis, herpes ocularis, chickenpox, shingles, zoster oticus, zoster varicellosus, keratitis, herpes digitalis, herpes facialis, herpes genitalis, herpes gladiatorum, or herpes stomatitis. More than one of any of the foregoing can characterize the herpetic disease afflicting the mammal.

In yet another aspect, compounds comprising pooled human immunoglobulins are disclosed. These compounds may be administered in any therapeutically effective manner including via parenteral administration, preferably intravenous injection, to a mammal in need thereof. The compounds are administered in a therapeutically effective amount, alone or in a pharmaceutically acceptable carrier to an animal. The animal in need of the compounds of the present invention either has HSV or is interesting in preventing an HSV infection.

The compounds comprising pooled human immunoglobulins can be used in any therapeutically effective manner including orally or parenterally by injection, drip, inhalation, or local administration and can be used alone, or as a pharmaceutical composition (for example, powders, granules, tablets, injections, emulsions, elixirs, suspensions, or solutions). Herein, a parenteral administration includes subcutaneous injection, intravenous injection, intramuscular injection, intraperitoneal injection and dripping infusion. A formulation for injection, for example, a sterile injection aqueous suspension or oily suspension can be prepared using a suitable dispersing agent or wetting agent and a suspending agent by a method known in the art. The sterile formulation for injection may be a sterile injectable solution or suspension in a diluent or a solvent which is non-toxic and can be administered parenterally, such as an aqueous solution. Examples of an acceptable vehicle or solvent which can be used include water, Ringer's solution and isotonic saline. As a solvent or a suspending solvent, a aseptic non-volatile oil can be also used usually. For such purpose, any non-volatile oil or fatty acid can be used, including natural or synthetic or semi-synthetic fatty oil and fatty acid, as well as natural or synthetic or semi-synthetic mono- or di- or triglycerides.

A compound comprising pooled immunologlobulins may be administered alone or in combination with other therapies against HSV and related conditions. A skilled clinician will be able to determine the appropriate amount of the compound to administer based on age, body weight, previous levels of administration and other factors.

The present invention also contemplates kits or packages that contain the pooled human immunologlobulins.

In another aspect, various animal models allow for testing IVIG therapies in vivo. An important strength of the model to explain IVIG protection is that the use of Rag/E strains has revealed facets of IVIG mechanism that would not be apparent if only WT mice were used; for example its ability to suppress HSV replication. The mouse models described herein are exemplary of the animal models that may be used to testing IVIG therapies.

### BRIEF DESCRIPTION OF THE DRAWINGS

FIG. 1 shows survival curves. Bone marrow transfers resistance and susceptibility. Mice were infected at 10×LD₅₀ HSV-1 17+ (circles), B6-E (middle line), 129BM (no irradiation); irradiated 129-BM (far left line with triangles).

FIG. 2 shows Fatal HSE is due to immune pathology. Infected mice were given daily IP injections of ACV (50 mg/kg; n=14) or PBS (n=12) from day 4-10 PI and monitored for mortality and signs of HSE. (A) ACV does not prevent or alter the kinetics of mortality. (B) ACV reduces HSV to undetectable levels in Tg and BS and (C) ACV does not affect the hyper-inflammatory response in BS; macrophage (CD45^{hi} F480−) and microglia (CD45^{int}) infiltrates remain elevated.

FIG. 3 shows HSV infection in 129-RAG/E and B6-RAG/E mice challenged with 3200 PFU HSV 17+.

FIG. 4 shows Passive immunization. (A) B6-Rag, 129-Rag and (B6×129) Rag-F1 mice and (B) Multiple treatments with human serum on day 1, 5 and 10 post-infection (PI).

FIG. 5 shows Passive immunization+ACV treatment. Mice were given antibody at 25 h PI and then treated with ACV from day 2-9 PI or day 4-11.

FIG. 6 shows IVIG reduces CNS inflammation: CNS mononuclear cells were isolated from BS (A and B) or brain (C and D) of infected B6, control 129 and 129 mice that received (129+IVIG) received 3 mg IVIG and analyzed for peripheral leukocytes by flow cytometric staining using anti CD45 Ab. Total CD45^{hi} infiltrates in B and D were calculated as % CD45 hi cells×total CNS mononuclear cells recovered as shown in A and C.

FIG. 7 shows that microglia from CNS of 129 mice are more activated than from B6 mice. Mononuclear cells were isolated from brain (left plot), spinal cord (middle plot) and BS (right plot) of C57B6 and 129 mice at d12pi. CD45^{int} F480+microglia from brainstem of B6 (shaded) and 129 (not shaded) mice were analyzed for MHC II expression as a measure of activation.

FIG. 8 shows differences in degranulation in macrophages in B6 and 129 spleen. Splenic macrophages (CD11b⁺) isolated from B6 (left) or 129 (right plot) at day 6 pi were incubated with CD107a Ab for 5 hrs. Shaded histograms represent isotype while CD107a reactivity is represented by histograms not shaded (A). Representative density plot of splenocytes derived from 129 mice at d6pi stained for Gr1⁻ and CD11b Ab (B). Gr-CD11b^{high} SSChigh neutrophils (middle row, right plot), Gr1⁻ CD11b⁻ immature macrophages (bottom row, left plot) and Gr1⁻ CD11b⁻ MHC 11⁻ macrophages (bottom row, right plot) were incubated for 5 hr with CD107a Ab with or without heat killed HSV Ag stimulation.

FIG. 9 shows IVIG maintains CD45^{high} infiltrates at low levels even at late times PI in all CNS compartments. Mononuclear cells isolated from BS and brain (not shown) of infected IVIG treated 129 mice at d35 p.i. were analyzed for peripheral leukocytes by flow cytometric staining using anti CD45 Ab (A). CD45⁻ cells, composed of both CD45^{high} infiltrating leukocytes, and CD45^{int} microglia, but not CD45⁻ neural cells, were further analyzed for F480 expression in (B) and MHC II expression in (C). CD45^{high} F480⁻ macrophages do not constitute the major CD45^{high} infiltrating population in BS (B) at d35 p.i. and do not appear to be recent emigrants as demonstrated by Ly6C^{int} reactivity (data not shown). Neither CD45^{high} F480⁻ macrophages (D), nor CD45^{int} F480⁻ microglia (C), express significant levels of MHC II suggesting a reduced state of activation. Accumulation of CD11c⁺ DCs with a B220+ plasmacytoid but not CD11b⁺ myeloid phenotype within the CD45^{hi} cells in BS (E and F).

FIG. 10(A) HSV in TG of 129 RAG mice is transiently controlled by HS treatment. Infected 129 RAG mice were administered IVIG on day 1, 12, 24 and 36 PI. Mice were observed for mortality and morbidity. (B) HSV titers in Tg of mice treated with IVIG on day 1, 12, 14 and 36 PI. **=titers in in vitro reactivated Tgs.

FIG. 11 shows IVIG limits inflammatory macrophages transiently. CNS CD45^{hi} infiltrates are reduced at both day 5 and day 8 PI in brain or BS of human serum (HS) treated 129 RAG mice compared to control 129 RAG mice (A). F480⁺ Gr-1^{high} macrophages observed in control mice were reduced at day 5 but not day 8 PI in CNS.

FIG. 12 shows adoptive transfer of CD4⁺ T cells into 129-Rag mice.

FIG. 13 shows IVIG protection in Gko Mice. (A) Survival of HSV infected Gko mice and (B) CNS inflammation: upper panels day 6 pi, lower panel day 18 pi.

FIG. 14 shows IVIG protects 129-E but not 129-Rag mice. HSV infected 129-E mice (A) or 129-Rag (B) were given HS or IVIG 24 PI and monitored for mortality.

FIG. 15 shows a model for IVIG action in HSV infected mice.

FIG. 16 shows protection by IVIG and its derivatives. Mice were treated at 24 h PI and monitored for signs of HSE. HSV−IVIG=IVIG lacking neutralizing antibody.

FIG. 17 shows Cu-64 counts in various tissues.

FIG. 18 shows dose dependent uptake of siCy3-RVG-9R by nerve cells.

FIG. 19 shows siCy3-RVG-9R taken up by nerve cells is cytoplasmic.

FIG. 20 shows RVG-9R delivers Cy3-siRNA specifically to PNS and CNS neurons in vivo.

FIG. 21 shows HSV negative IVIG protects from fatal HSE. Pooled sera obtained from 30 HSV negative donors was given to HSV infected 129 mice 24 hours PI. Morality or morbidity was observed. Data represents 3 experiments (n=21).

FIG. 22 shows transferred IVIG primed S-cells or Tregs protect wt 129 from HSE. (A) 129 mice transferred with splenocytes or Tregs from IVIG treated mice and (B) mice transferred with in vitro IVIG primed or control BMDCs. Non-transferred control mice received only PBS in both A & B.

FIG. 23 shows brain stem inflammation is independent of virus clearance. CD45^{high} infiltrating cells were analyzed in 129 BS at indicated time points post HSV infection by flow cytometry. Infectious virus titers were determined in BS of infected mice by plaque assay. Data is representative of 3 experiments with 3-4 mice per time point. >90% of mice die by day 12 pi.

FIG. 24 shows alternatively activated macrophages. M1 and M2 macrophages: the extremes of a continuum. Macrophage activation is associated with profound changes in gene expression profiles. Exposure to different tissue-derived stimuli induces distinct polarization profiles, associated with the expression of selected molecules. Classical macrophage activation (M1 macrophage) is induced by exposure to IFN-g and LPS, and it is associated with a distinct set of molecules (red). Different forms of alternative activation (M2 macrophage) can be due to different stimuli, with distinct molecular profiles. IL-4 and IL-13 induce M2a (yellow), immune complexes CLPS induce M2b (pink), and IL-10 induces M2c (green). Molecules in common for M2a and M2c (induced both by IL-4CIL-13 and IL-10) are in blue.

### DETAILED DESCRIPTION

In one aspect, the invention provides a new method for treatment and prevention of HSV induced disease by intravenous administration of pooled human IgG (IVIG).

Fatal HSV encephalitis (HSE) results from hyper-inflammatory responses in the CNS of susceptible mice rather than from direct virus cytopathology. Furthermore, Acyclovir (ACV), the drug recommended for treatment is largely ineffective in protecting against fatal HSE despite efficiently suppressing viral replication. Human IVIG (pooled IgG for intravenous injection) protects 100% of susceptible 129 mice from fatal HSE. Studies using immunodeficient mice demonstrate IVIG can protect both susceptible 129 and resistant C57BL/6 (B6) immunodeficient mice form HSV induced death; protection of 129 mice depends on suppression of inflammation and HSV replication, whereas protection of B6 mice involves only suppression of viral replication since these resistant mice do not have hyper-inflammatory CNS responses after HSV ocular inoculation. The present discovery reveals that IVIG can suppress intracellular replication of HSV, which is unprecedented.

Significant progress has been made in defining the mechanism(s) of protection of IVIG including that it induces Tregs and pDCs that accumulate in the CNS of infected mice. The active component of IVIG has been defined as a sialylated monomeric IgG species, specifically just the Fc as mediating IVIG's anti-inflammatory activity and a role for HSV specific antibodies including neutralizing antibodies was excluded. Comparative studies of protection against HSE in wt 129 and 129-Rag strains revealed that a cell type absent in the Rag mice mediates robust long-term protection in wt mice, called the ‘P-cell’. The cell is CD3+ lymphocyte and CD4+ and CD8+ T cells were excluded, leaving NKT and gd T cells as candidates.

The present invention describes that IVIG treats HSE, herpes stromal keratitis (HSK), and other HSV-related diseases or disorders. Like HSE, HSK is immune-mediated hence treatment with ACV is not particularly effective and has the complication of promoting ACV resistant mutant strains. Additionally, IVIG has the potential to suppress reactivated HSV and hence maybe a useful intervention for individuals suffering from frequent HSV reactivation, including genital HSV-2 infections. Additionally, West Nile Virus encephalitis (WNV) involves an immunopathological component, hence, the mechanism by which IVIG protects against WNV involves its anti-inflammatory activity rather than just neutralizing activity. Moreover, IVIG can treat Japanese Encephalitis Virus (JEV) and Venezuelan Equine Encephalitis Virus (VEEV) and other encephalitis conditions with immunological bases.

The Fc-domain of sialylated IgG mediates IVIG's anti-inflammatory activity. The IgGs are isolated from humans and reproduced using any practical method, including the techniques described by Harlow E & Lane D (1999) in Using Antibodies: A Laboratory Manual. Cold Spring Harbor, N.Y.: Cold Spring Harbor Laboratory Press and/or using genetic engineering. Additionally, the cell that initially responds to IVIG and transduces its immunomodulatory effects, the so called ‘Sensor cell’, is a dendritic cell (DC). The receptor for sialylated IgG is distinct from the FcgR receptors and identifying this receptor on DCs provides an alternative means of activating the S-cell leading expression of immunodulatory functions.

As used herein, the term “effective amount” means an amount sufficient to produce a therapeutic result. Generally, the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition.

Unless otherwise indicated, the term “purified” means that a substance has been removed from its original environment, such that the relative abundance of the substance has been increased in the context of other components of the composition. Alternatively or additionally, the term can be used solely to indicate that at least one particular contaminant has been reduced or removed from the composition comprising the substance.

The term “pharmaceutically acceptable carrier” refers to any compatible non-toxic material suited for mixing with the active compounds of the present invention.

The phrase “prepared from pooled plasma” means that the immunoglobulin preparations modified by this phrase were recovered from pooled plasma originating from blood donors. The compositions of the invention do not require that such donors be screened to determine their levels of HSV-neutralizing antibody.

Intravenously administered compositions of the invention can be used for the treatment and/or prophylaxis of disseminated infections, thereby preventing herpes-related disease such as herpetic encephalitis. Such compositions can be used for the treatment and/or prophylaxis of neonatal infections, thereby treating or preventing disseminated infections that currently generate a high mortality rate. Immunocompromised individuals, including neonates and the elderly, are particularly susceptible to such diseases and benefit from administration of the compositions of the invention. the dosage of the immunoglobulin compositions of the invention and the method of administration will vary with the severity and nature of the particular condition being treated, the duration of treatment, the adjunct therapy used, the age and physical condition of the subject of treatment. However, single dosages for intravenous administration can typically range from about 50 mg to about 10 g per kilogram body weight, or from about 100 mg to about 5 g per kilogram of body weight (unless otherwise indicated, the unit designated “mg/kg” or “g/kg”, as used herein, refers to milligrams or grams per kilogram of body weight).

The quantity of active component, e.g. an immunoglobulin preparation of the invention, in a pharmaceutical composition can be varied or adjusted widely depending upon the requirements of the patient, the severity of viral infections, the potency of the particular compound being used, the particular formulation and the desired concentration. Generally, the quantity of active component will range from about 0.05% to about 25% by weight of the composition, from about 0.1% to about 10% by weight, or from about 3% to about 16% of the composition.

### Examples

Peripheral infection of the mouse is an amenable model for the study of HSV pathogenesis. Experimental variables such as virus virulence, route of inoculation, age and sex of mice are easily controlled. Inbred mouse strains vary greatly in their sensitivity to HSV-1 infection, with B6 being the prototypically resistant strain (LD₅₀>10⁵ PFU) and 129S6 (129) and BALB/c, examples of susceptible strains (LD₅₀ 1-5×10² PFU). A 10×LD₅₀ dose of virus (for the 129 strain, 3×10³ PFU) results in 90-100% mortality for susceptible mice, compared to 0-15% mortality for resistant mice (68). In all studies reported below mice were challenged with a 10×LD₅₀ dose of HSV-1 strain 17+ determined in 129 mice unless otherwise indicated.

Immune Mechanisms in Fatal HSE.

Using a novel B6-E mouse strain (B6.IL-7R^{-/-}Kit^{w41/w41}) as recipient for bone marrow (BM) transplant (BMT)(26), it was shown that resistance and susceptibility to HSE are transferable genetic traits. The dominant negative Kit^{w41/w41} mutation affects primarily the kinase but not other aspects of the c-Kit receptor (92). The B6-E mouse lacks B, T, NKT and gdT cells as a result of IL-7R deficiency and the Kit^{w41/w41} mutation (33, 51, 81, 99) but NK cell levels are normal. Kit mutations additionally interfere with mast cell (MC) development and function and Kit^{W/W-v} mice are profoundly deficient in tissue mast cells (59, 75, 92, 106). Mast cells could be grown out of BM from B6, 129 and B6-Rag but not BM from B6-E mice using media supplemented with Kit ligand and IL-3 (not shown), which indicates a mast cell developmental defect in the B6-E strain.

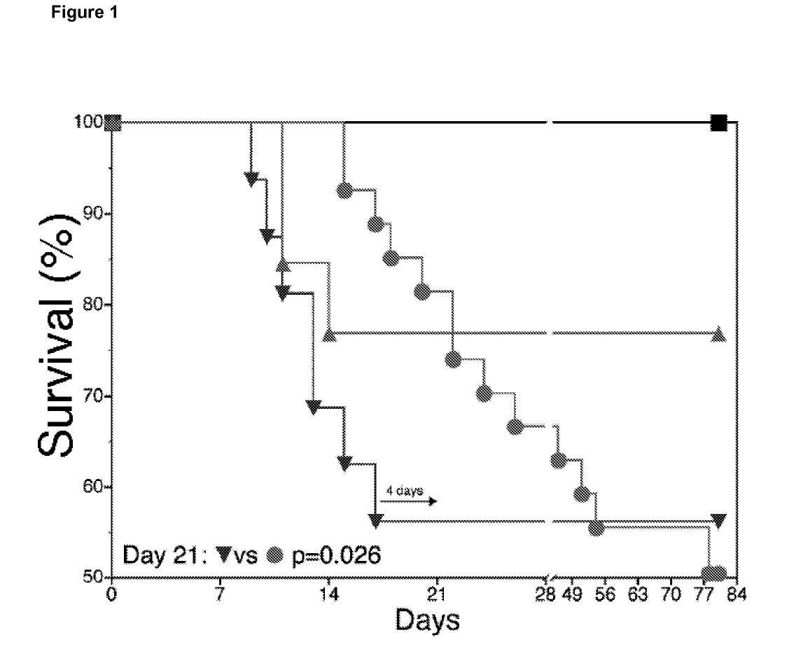

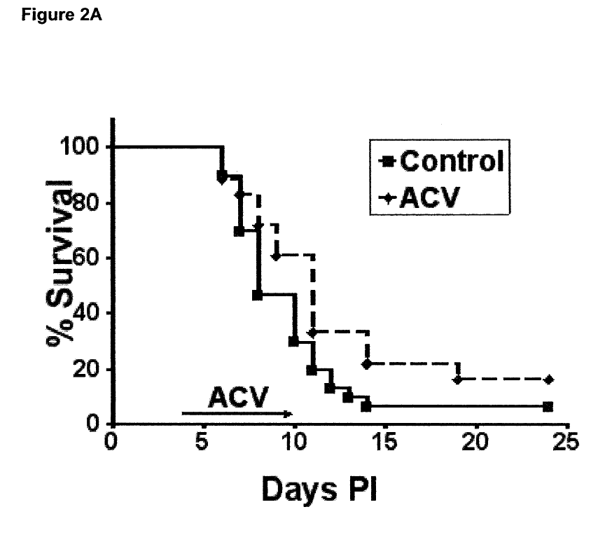

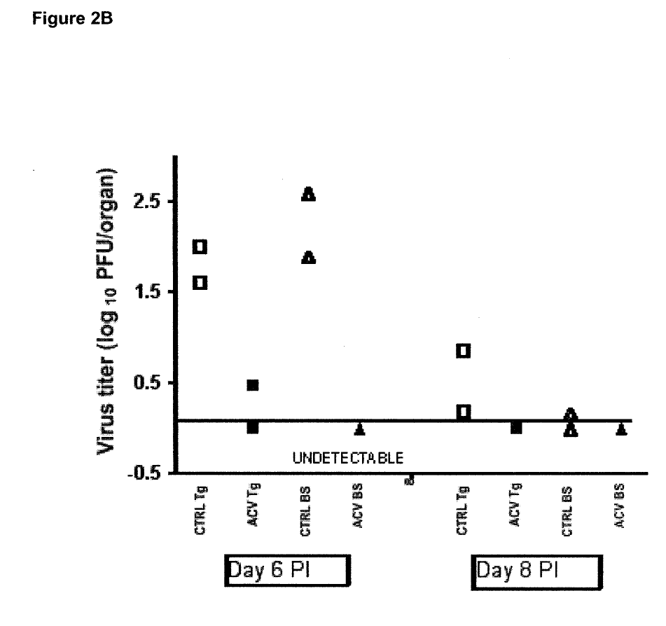

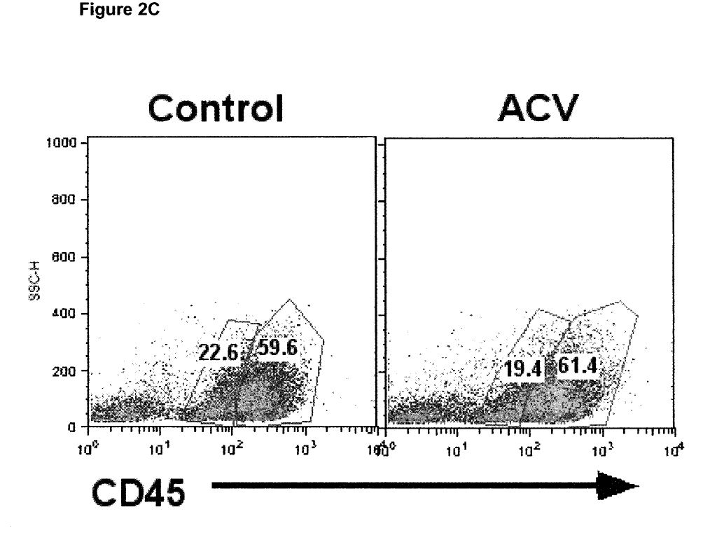

6 week old B6-E mice, either lethally irradiated (to ablate endogenous innate cells) or non-irradiated, were reconstituted with 129- or B6-BM. At 10 weeks engraftment was assessed by flow cytometry of blood samples for B and T cells. At ˜12 weeks the different mouse groups were blind challenged with HSV-1. 100% of mice transferred with B6-BM survived irrespective of radiation conditioning. B6-E mice receiving 129-BM (129-BMT) showed variable mortality; 22% for non-irradiated recipients compared to 46% for irradiated recipients. Remarkably, compared to non-BMT control B6-E mice, 129 BM actually accelerated the onset of death; at the time the last 129-BM recipient died (d15), practically all control non-BMT B6-E mice were still alive (FIG. 1). This shows that an excessive inflammatory response to HSV infection in susceptible 129 mice contributes to fatal HSE, a conclusion bolstered by our recent studies of CXCR3 signaling in these strains (67). The slower kinetics of mortality for the B6-E mice suggests a crucial role for innate immune responses in protection against HSE, also supported by the difference between the 129-BM recipients that were irradiated versus those that were not (FIG. 1). Remarkably, in this experiment 50% ( 13/26) of non-BMT control mice survived infection for >77 days. FIG. 2 shows that although the antiviral drug ACV (given from day 4-10 P reduces HSV to undetectable levels in the Tg and BS it fails to prevent death due to hyper-inflammatory responses in the BS. ACV treatment was delayed to allow the HSV to reach the BS and replicate as this is essential to initiate inflammation. These studies establish that the pathogenic nature of 129-derived BM immune responses in HSV infection.

### Differences in Innate Immunity in B6 and 129 Mice.

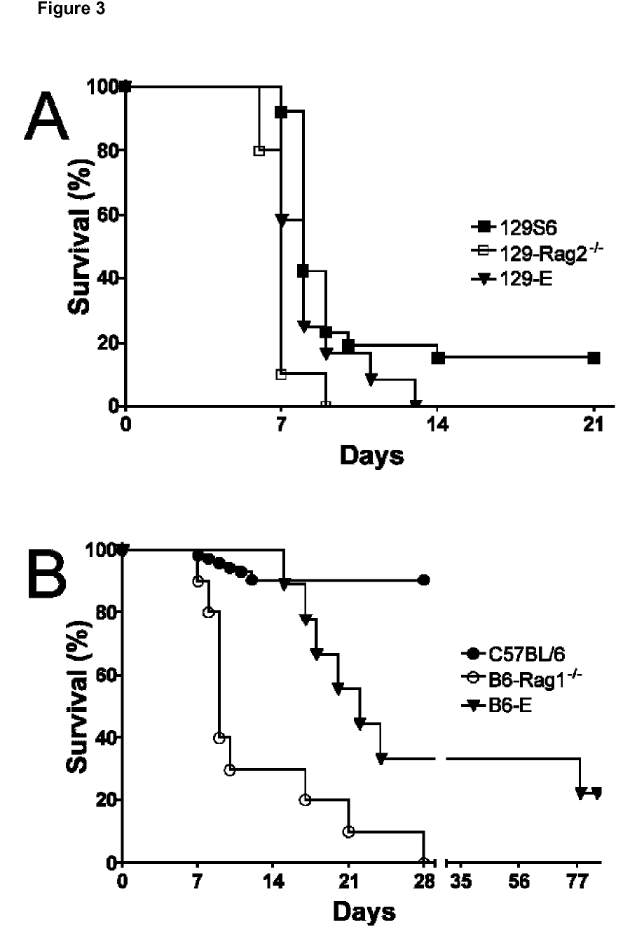

Resistance to fatal HSE is inherited as a dominant trait since (B6×129)F1 mice are resistant (68). B6-E mice lack adaptive immunity yet they are significantly more resistant than wild type 129 mice (FIG. 3). HSV infection in B6-Rag1 was compared with 129-Rag2 mice that lack T, B and gd T cells; the Rag1 and Rag2 mutations are functionally equivalent and in subsequent studies we utilized B6-Rag2 and 129-Rag2 mice. Survival curves showed both RAG strains were more susceptible than the wild type strains to HSV, (p<0.0001), but the difference was accentuated for resistant B6 mice (FIG. 3). B6-Rag mice were more resistant than 129-Rag mice (p<0.0007). Unexpectedly, B6-E mice were significantly more resistant to HSE than either B6-RAG (p<0.009) or 129-RAG (p<0.0001) mice at day 7 PI and 129-E mice were also more resistant than 129-Rag (p=0.0260) (FIG. 3). Since, compared to Rag mice, E mice additionally lack mast cells this suggests that mast cells are deleterious in the absence of B & T cells (FIG. 3B). Precedent for this assertion is the report that mast cells are primarily responsible for inflammation in the brain following Sindbis virus infection (84). By inoculating groups of mice with 20, 100 and 500 PFU HSV and calculating survival proportions at each challenge dose it was estimated that LD₅₀ for 129-Rag2, B6-Rag2 and the corresponding F1(129-Rag×B6-Rag) as ˜30 PFU, 100 PFU and 60 PFU, respectively (FIG. 1). It is evident that the differences in resistance will be maximal at about day 10 PI with an inoculum of 100-500 PFU (FIG. 1). Resistance is dominant for the wild type strains whereas resistance of Rag (129×B6)F1 is intermediate between the B6-Rag and 129-Rag strains. These results motivated construction of the 129-E strain, which is more resistant than the 129-Rag strain again implicating a role for mast cells (FIG. 2). The 129/B6-E strains will facilitate comparison of mast cell effects in HSV infection and in particular, their involvement in immune pathology in 129 mice.

### Passive Immunization Protects B6-Rag and B6-E Mice but not 129-Rag Mice.

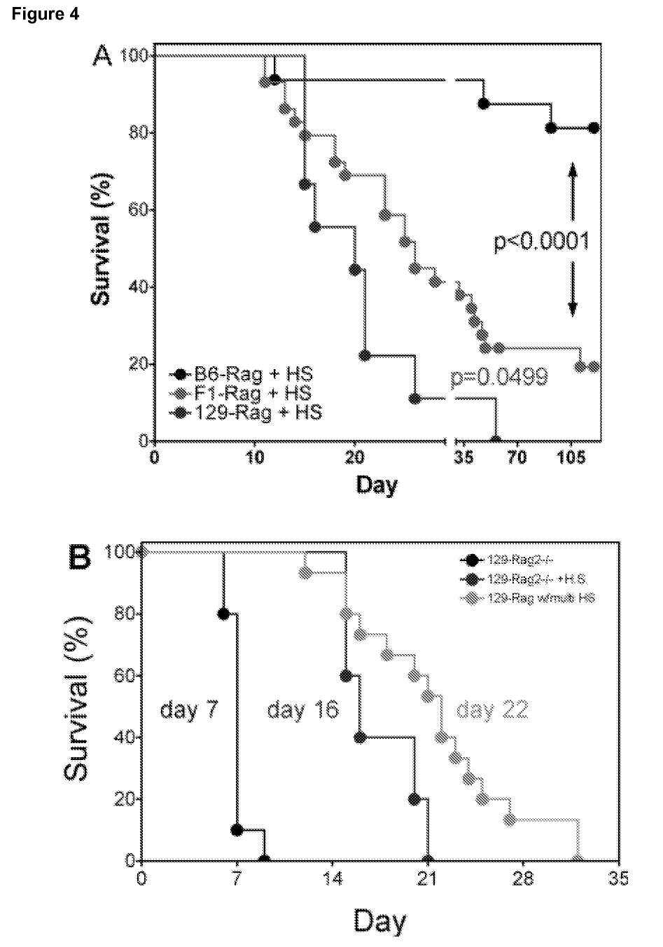

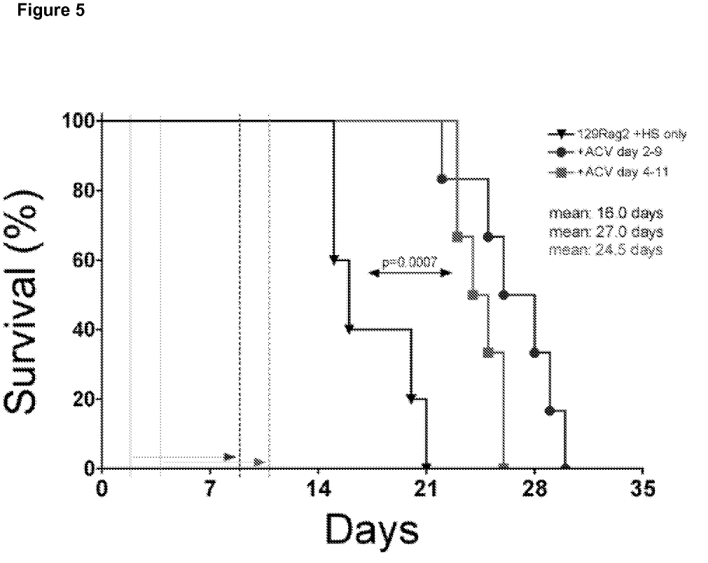

B6-Rag and 129-Rag mice die after HSV challenge. Passive immunization with pooled human serum (HS) was evaluated as a means to promote survival with normal latency as has been done with various wild type strains. Mice were given 500 ml heat inactivated HS IP at 24 h PI, a dose previously reported to protect 100% of WT mice (22). HS protected >85% of B6-Rag mice in comparison to none of the 129-Rag mice (p<0.0001), all but one dying by day 28 PI. Unexpectedly survival of (129×B6)Rag-F1 mice was only marginally increased relative to 129-Rag mice (p<0.0499) (FIG. 4A). 100% of wild type 129 mice were protected as expected (not shown). Next, multiple injections of HS were given on day 1, 5, and 10 PI and though this prolonged survival slightly, all the 129-Rags eventually died (FIG. 4B). These results led to speculation that HS treatment might have resulted in persistent infection of 129-Rag mice. To evaluate combination HS+ACV treatment, 129-Rag mice were given a single IP injection of human serum at 24 h PI and treated with ACV on days 2-9 PI (group 1, 7 mice) or days 4-11 PI (group 2, 8 mice). ACV was injected IP at 50 mg/Kg on day 2, 3 PI, followed by ACV in the drinking water (2 mg/ml) from day 3-9 PI for group I mice, while group II mice received ACV injections on day 4, 5 PI followed by ACV in drinking water from day 5-11 PI. The mean survival time for mice treated with antibody only was 16 days and though mean survival time was significantly increased with ACV treatment to 27 days (group I) and 24.5 days (group II) (p=0.0007 in comparison to no ACV treatment), all the 129-Rag mice eventually died (FIG. 5). Thus, HS+ACV given on day 2-9 versus on day 4-11 was more effective in controlling HSV replication and prolonging survival as indicated by the earlier onset of death in group II mice (ACV d4-11) (FIG. 5). Modest amounts of virus were found in the BS and Tg of dead mice. Results are typical of several experiments that differed slightly in the timing and duration of ACV treatment, but overall the outcome was always the same—survival was extended but ultimately all 129-Rag mice died from fatal HSE.

Important conclusions are: (i), B6-Rag mice are much more susceptible than B6-E mice, which implicates mast cell responses as being deleterious in B6-Rag mice (ii), HS cannot protect 129-Rag mice even when combined with extended ACV treatment (iii), a cell type absent in Rag mice (i.e., B, CD4+ and CD8+, gd and NKT cells) is essential for HS mediated survival because all HS treated WT 129 mice survive lethal HSV challenge (iv), neurons (and perhaps associated glia) in 129 and B6 mice differ fundamentally in their capacity to restrict HSV replication resulting in survival with establishment of latency, because early passive immunization and ACV treatment essentially confines HSV to neurons precluding spread of infection.

### Anti-Inflammatory Effects of IVIG in WT 129 Mice.

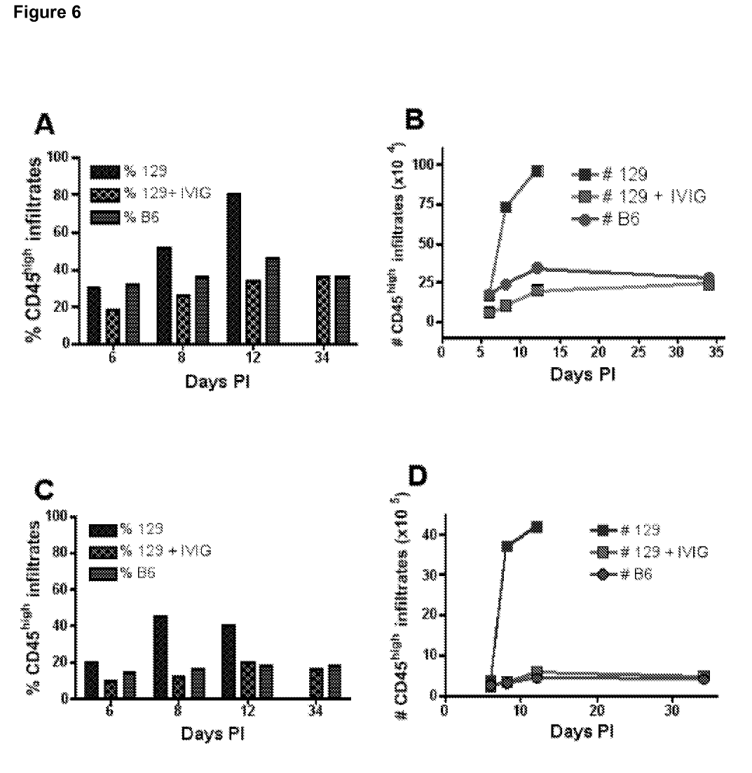

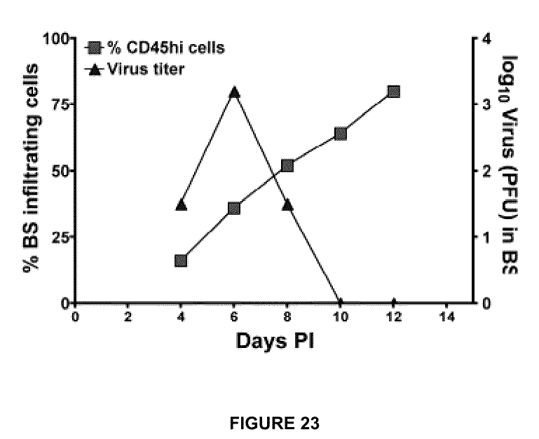

Analysis of CNS infiltrates in IVIG treated and control 129 mice following HSV infection revealed a striking reduction of CD45^{high} infiltrating peripheral mononuclear cells in treated mice. FIG. 6A shows increased (5 fold) CD45^{high} infiltrates in control mice between days 6-8 PI compared to BS of IVIG recipients that show only a 2 fold increase. At day 8 PI, 7-fold more CD45^{high} infiltrating cells were present in BS of control mice compared to IVIG recipients (FIG. 6A). Increased CNS infiltration is evident still at day 12 PI in the few surviving control mice (FIG. 6A). At day 6 PI similar inflammation is seen in BS of B6 mice compared to 129 mice but while inflammation progresses unabated in the BS of 129 mice through d12 pi, it is greatly diminished in the BS of B6 mice at days 8-12 PI (FIG. 6A). When presented as total infiltrating cells the differences in recruitment of cells into the BS are even more dramatically emphasized (FIGS. 6B & D). Another important finding is that IVIG treatment limited inflammation to the BS. In contrast, leukocytes infiltrated the brain, brainstem and spinal cord of control mice robustly by day 6-12 PI (FIG. 6C) resulting eventually in fatal HSE. There is at least a 10-fold increase in CD45^{high} infiltrates in brains of control mice compared to IVIG recipients (FIG. 6C). These data indicate that 129 mice have an intrinsic defect in regulation of inflammation in the CNS. A single IVIG treatment confers on 129 mice the ability to regulate CNS inflammation and thereby prevents destructive inflammatory responses similar to that observed in B6 mice. Another observation is that CNS inflammation was independent of HSV clearance (FIG. 23). These data indicate that 129 mice have an intrinsic defect in regulation of inflammation in the CNS. A single IVIG treatment conferred on 129 mice the ability to regulate CNS inflammation and thereby prevented destructive inflammatory responses.

### Macrophages are Pathogenic Effectors in 129 Mice.

A major difference between B6 and 129 mice is the extensive infiltration of leukocytes into the CNS of HSV infected 129 mice. Macrophages constituted the predominant infiltrating population in BS of infected mice and together with Gr-1⁺/Ly-6G⁺ neutrophils comprised the majority of cells in BS at day 6 pi (Table 1). However, by day 8-12 pi, in contrast to 129 BS, macrophages were not the dominant population in the B6 BS or BS of 129 mice treated with IVIG (Table 1).

TABLE 1
Composition of CD45^{high} infiltrating leukocytes into the BS of
B6, control 129 and IVIG treated 129 mice at d 6 and d 12 PI.

| | B6 | 129 | 129 + IVIG |
|---|---|---|---|
| Day 6 pi | | | |
| Macrophages (CD45^{hi}, F480+, Ly6G−) | 58* | 65 | 56 |
| Neutrophils (CD45^{hi}, Ly6G+) | 6 | 18 | 6 |
| CD8 T cells | 4 | 8 | 8 |
| CD4 T cells | 5 | 6 | 10 |
| Day 12 pi | | | |
| Macrophages (CD45^{hi}, F480+, Ly6G−) | 20 | 50 | 35 |
| Neutrophils ((CD45^{hi}, Ly6G+) | 2 | 15 | 2 |
| CD8 T cells | 30 | 18 | 25 |
| CD4 T cells | 25 | 15 | 20 |

*All numbers represent percentages within the CD45^{high} infiltrates.

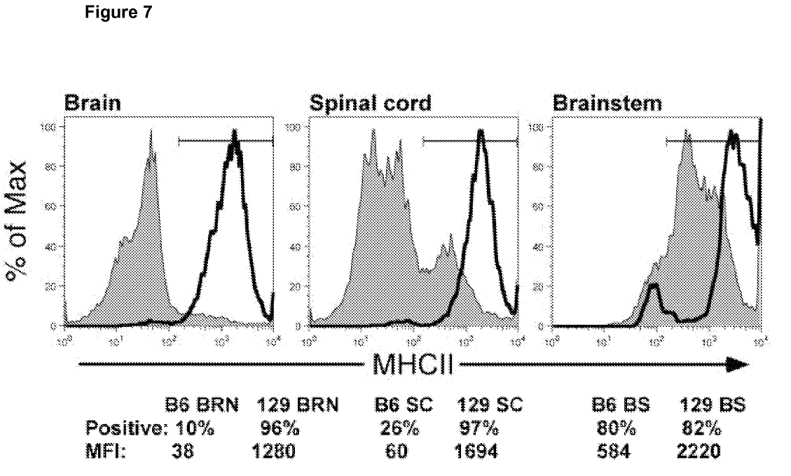

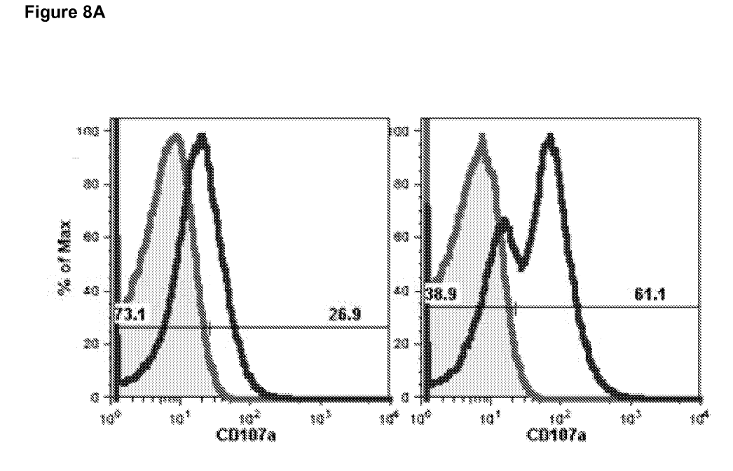

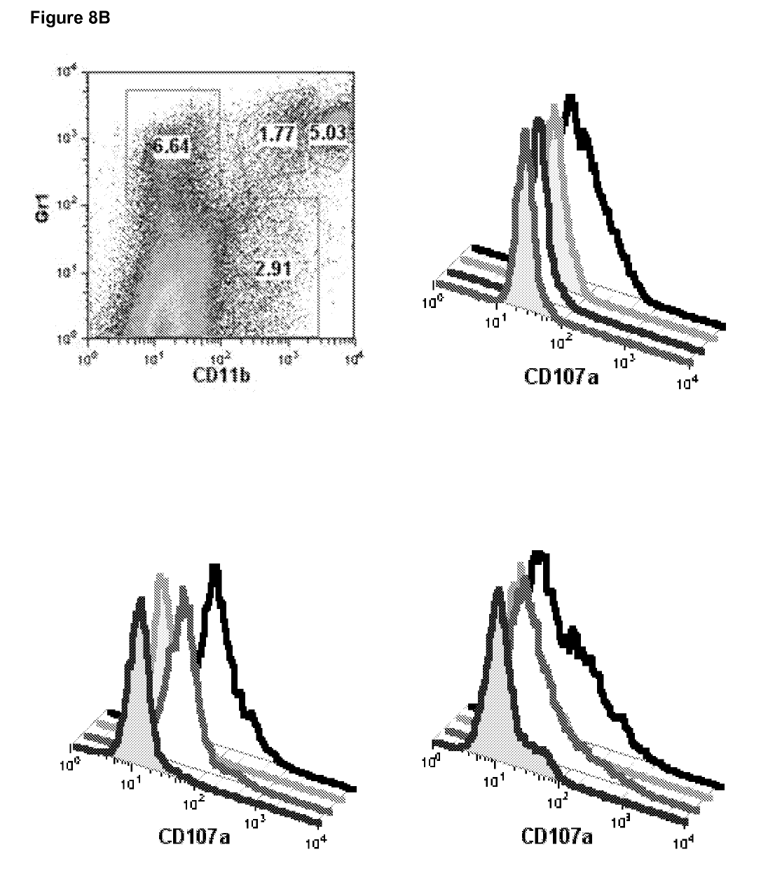

Inflammation was widespread in all CNS compartments of 129 mice but restricted to BS and less severe in B6 mice (shaded) as reflected by the contrasting MHC-II expression on microglia (FIG. 7). A similar trend was observed in IVIG treated 129 mice wherein microglia from BS but not other CNS compartments expressed MHC II molecules (data not shown). Thus the capacity of IVIG to limit inflammation in 129 CNS achieves the same effect as the control mechanisms that normally regulate inflammation in HSV infected B6 mice. Macrophages isolated from spleens of infected 129 mice displayed high level of spontaneous degranulation even in the absence of any stimulation as determined by surface CD107a expression by flow cytometry analysis (FIG. 8A, right plot histogram). However, macrophages isolated from infected B6 mice did not degranulate spontaneously (FIG. 8A, left plot). This result is consistent with the observed lack of spontaneous degranulation by splenic macrophages isolated from infected IVIG treated 129 mice that also showed reduced degranulation following HSV Ag stimulation compared to 129 macrophages. Furthermore, the subset that degranulated maximally in 129 splenocytes was determined to be Gr1⁺ 11b⁺ immature macrophages (FIG. 8B, bottom row, left plot). However, Gr1⁺ 11b^{high} SSC^{high} neutrophils (FIG. 8B, middle row, right plot) and Gr1⁻ 11b⁺ MHC II⁺ mature macrophages (FIG. 8B, bottom row, right plot) showed reduced degranulation in the absence of Ag stimulation. However, both these subsets displayed increased degranulation when stimulated with heat killed HSV Ag.

In vivo depletion of macrophages in 129 and B6 mice revealed pathogenic and protective roles, respectively in these strains (69). Consistent with this finding, these results implicate the divergent responses of macrophages in these two strains of mice as major determinants of the outcome of HSV infection. Macrophages in the CNS of 129 mice also express higher levels of activation markers compared to those in CNS of IVIG treated mice (FIG. 2).

### Long-Term Protective Effects of IVIG in WT Mice.

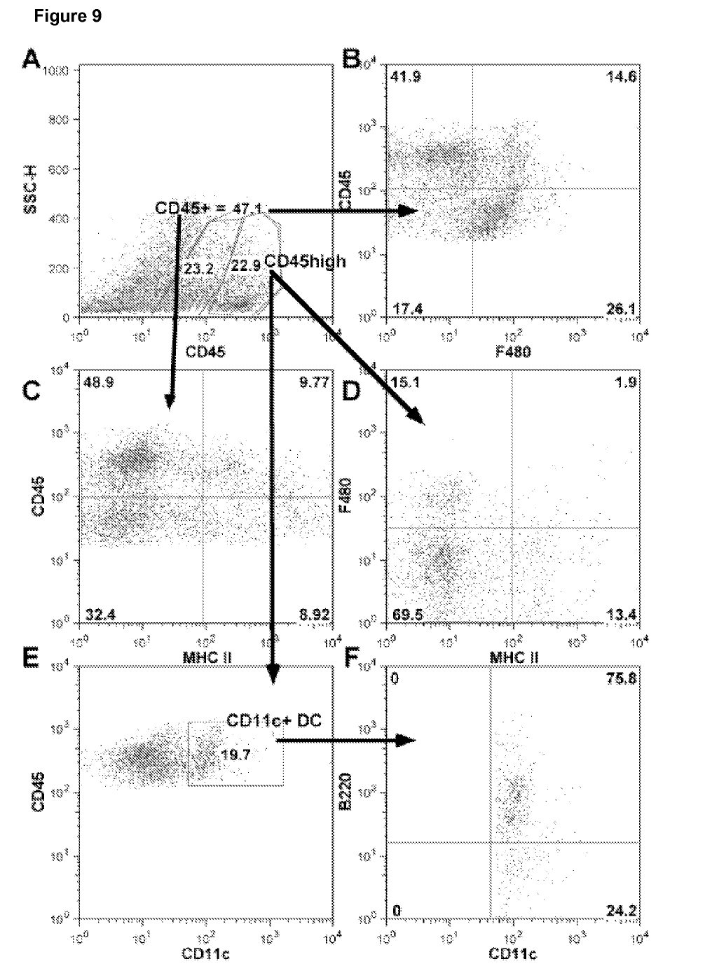

The mechanism by which IVIG protects 129 WT mice likely revolves around its ability to induce long-term protective anti-viral responses as well as its capability to prevent and modulate deleterious inflammatory responses. Analysis of infiltrates in brain and BS of IVIG treated 129 WT mice revealed that the initial constraints in accumulation of peripheral infiltrates persisted with 23% CD45^{hi} infiltrates detected at day 35 PI (FIG. 9A). This restraint was also reflected in the CD45^{hi} F4/80⁺ macrophage population, which comprised about 25% of CD45^{hi} infiltrates with relatively few recent (Ly6Chi) emigrants. Overall reduced CNS inflammation in these mice was also reflected in both brain and BS by the diminished MHC II expression by CD45^{int} microglia (FIG. 9C) and CD45^{hi} F480 macrophages (FIG. 9D). CD11c⁺ DCs with a plasmacytoid B220⁺ PDCA1⁺ phenotype however constituted a significant population within CD45^{hi} cells in BS (FIGS. 9E and F) and brain and their presence potentially implicates a regulatory role for DCs in the down-regulation of deleterious macrophage responses. Control PBS treated infected 129 mice do not survive past day 12 PI.

In conjunction with the diminished inflammatory responses observed in the CNS of IVIG treated mice, infectious virus could not be detected in the TG, BS or brain at day 35 PI. The composition of infiltrates in the CNS was also altered from the characteristic predominance of granulocytes and monocytes detected in 129 control mice to a population containing both monocytes and lymphocytes. Overall, these results indicate that an intricate balance is established following IVIG treatment wherein suppression of both pathogenic macrophages and replicating virus results in a beneficial outcome to the host.

### Transient Anti-Inflammatory IVIG Effects in 129 RAG Mice.

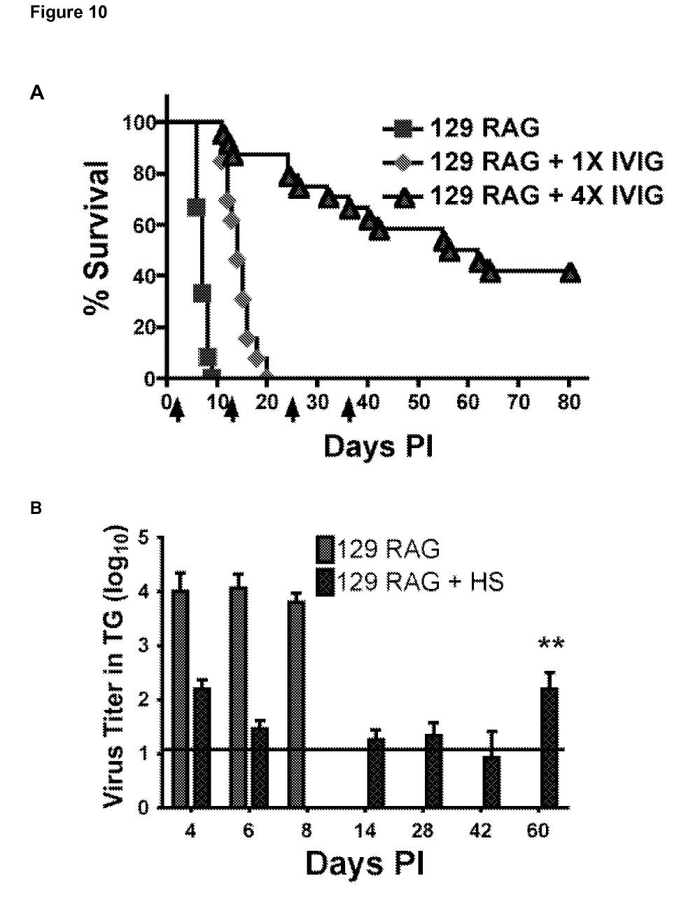

The ability of IVIG to control HSV pathogenesis in 129 WT mice but only transiently in 129 RAG mice potentially implicates a cell subset missing in Rag mice in regulating the expansion of effector macrophages as well as being involved in suppression of virus replication. Despite significant improvement in survival, IVIG administered at 12-day intervals (d1, 12, 24 and 36 PI) could not completely protect 129 RAG mice infected with 3200 PFU (FIG. 10). However, virus was undetectable in Tgs of mice sacrificed at >greater day 20 after the last IVIG treatment at day 36 P, although latent HSV was present as determined by in vitro reactivation; indicated by ** in FIG. 10B. Following a single injection, IVIG controlled virus replication by day 6-8 PI in 129 RAG mice as evidenced by the absence of infectious virus (Table 2). Although CD45^{high} infiltrates were significantly reduced in the brain and BS of IVIG treated 129 RAG mice (˜10%) compared to control mice (˜35%) at day 5 PI, this control of inflammation was progressively lost as evidenced by the increased infiltration at day 8-14 PI in the IVIG treated mice correlating with reactivating virus in the Tg (Table 2). Virus was transiently controlled following each IVIG dose with ˜40% of RAG mice surviving after receiving 4 doses of IVIG (FIGS. 10A and B).

TABLE 2

| | |
|---|---|
| 129 RAG | 129 RAG + 1 dose HS^{a} |

| | | CD45^{hi} | | CD45^{hi} |
|---|---|---|---|---|
| Days PI | TG Virus^{b} | cells | TG Virus | cells |
| D 5 | 4.1 ± 0.6 | 34% | 1.5 ± 0.4 | 10% |
| D 7 | 3.8 ± 0.3 | 30% | ND* | 14% |
| D 14 | NA** | | 1.2 ± 0.5 | 22% |

^{a}One group of mice received pooled human IgG 24 hours post HSV infection.
^{b}Virus titers in TG depicted as log₁₀ PFU.
*ND: Not detectable,
**NA: mice are dead by D8-10 PI.

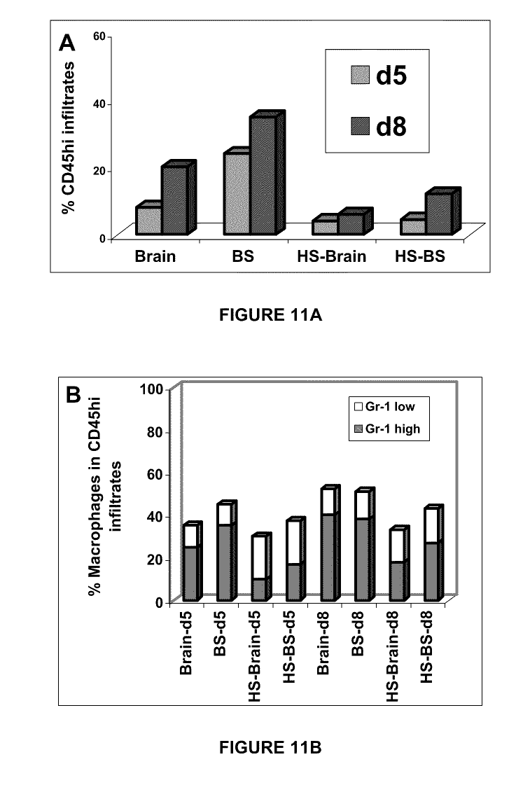

The major cell type infiltrating the brain and BS of 129 RAG mice were F480⁺ macrophages that expressed high levels of the Gr-1 marker (FIG. 11B). Macrophages bind the Ly-6C component of the Gr-1 Ag and Ly-6C+ monocytes are considered as recent tissue emigrants while those that express low levels of Ly-6C/Gr-1 are considered more mature macrophages. Gr-1 reactivity also correlates with an aggressive phenotype. While >80% of macrophages isolated from brain and BS of control mice expressed high levels of Gr-1, only ˜40% of macrophages present in the CNS of IVIG treated mice were Gr-1^{high} at day 5 PI. This data correlated with the reduced aggressive phenotype of these cells. However, by day 8 PI increased infiltration of macrophages in IVIG treated mice was reflected in increased numbers of Gr-1^{high} macrophages in conjunction with CD45^{high} cells in the CNS (FIGS. 11A & B). These data demonstrate the transient IVIG mediated protection afforded in 129 RAG mice wherein expansion of the macrophage population and suppression of virus replication is not sustained.

### IVIG Does not Require HSV Specific IgG to Induce Protection in Wt 129 Mice.

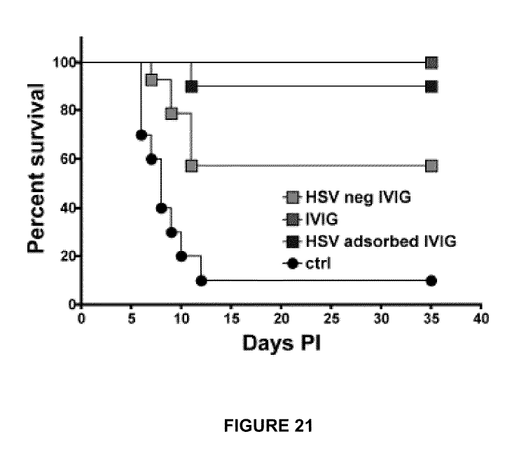

Serum collected from 30 HSV seronegative donors was pooled and given to HSV infected wt 129 mice 24 h pi. This Ab preparation protected >60% of mice (FIG. 21) consistent with IVIGs anti-inflammatory effects being independent of HSV specific IgGs. Reduced protection by HSV negative IVIG compared to IVIG devoid of HSV neutralizing Ab is due to a lower concentration of sialylated IgGs in the HSV negative IVIG preparation. Analysis of sialylated IgG levels in the two preparations is in progress.

### IVIG Treatment of BMDCs Alters the Responsiveness to TLR Ligands.

Bone marrow derived DCs (BMDCs) generated from 129- and B6-Rag mice were treated with IVIG for 24-48 hours prior to exposure to LPS (100 ng/ml). 24 h later, mRNA was prepared from these cells and analyzed for type I IFN, TGF-b, IL-10 and TNF-a. TNF-a did not increase drastically in most preparations. Studies have shown that LPS exposed BMDCs secrete TNF-a maximally from 6-12 hrs after which TNF-a secretion is reduced. TNF-a levels were greater in control BMDCs stimulated with LPS (140 fold increase over control untreated DCs) compared to IVIG treated DCs (7 fold increase). IFN-a levels were also increased (Table 3) in LPS treated control DCs (68 fold) compared to IVIG treated DCs exposed to LPS (7 fold). In contrast, expression of TGF-b and IL-10 were greatly increased in both IVIG treated DCs and IVIG treated DCs exposed to LPS.

Treatment of DCs with IVIG for 48 h increased TGF-b and IL-10 expression levels by 485 and 170 fold respectively, compared to control untreated DCs. LPS exposure for an additional 24 h further enhanced expression of TGF-b (1553 fold) but not IL-10 (25 fold). LPS inhibited TGF-b secretion by control untreated DCs (0.05 fold) while IL-10 was moderately increased (65 fold). A very similar pattern emerged for BMDCs derived from B6-Rag mice (not shown) indicating that the effect of IVIG in generating a type 2 DC phenotype may be independent of mouse strain background. As reflected in Table 3, IVIG modulates response to the prototypical TLR ligand LPS in BMDC and we suspect other TLR ligands, which is a novel finding with important clinical implications. IVIG and LPS both induce IFN-a in BMDC but LPS fails to induce IFN-a in IVIG primed BMDC. During HSV infection DCs are primed primarily, if not exclusively, by cross presentation and IFN-a is critically required, hence by blocking IFN-a induction by TLR ligands IVIG may moderate activation of HSV specific T cells.

The ability of IVIG to modulate responsiveness to LPS was less impressive when surface MHC II and CD86 expression was compared in untreated and IVIG treated DCs exposed to LPS. LPS treatment increased surface MHC II expression and CD86 (not shown) dramatically for control DCs as depicted by mean fluorescence intensity (MFI). Unexpectedly, IVIG treated DCs exposed to LPS also showed significant up-regulation of both MHC II and CD86 expression though not as striking as with the control DCs. These data therefore suggest that IVIG treated DCs retain the ability to present antigen to T cells and may be efficient APCs. However, while LPS treated control DCs have been shown to induce T cells to a Th1 pathway, IVIG treated DCs may be efficient generators of Th2/Treg cells as they express high levels of IL-10 and TGF-b. These results concur with our in vivo results wherein IVIG treatment increased Treg generation. These data thus suggest that DCs treated with IVIG can present antigen efficiently but rather than generating Th1 responses, instead generate Th2 responses and Treg cells qualifying them as the sensor cell (s-cell) that initially responds to IVIG.

TABLE 3
Ratio of cytokine expression following LPS
treatment in IVIG treated BMDC compared to
control BMDC derived from 129 Rag mice.

| Cytokine | IVIG¹ | IVIG + LPS | LPS |
|---|---|---|---|
| IFN-α | 48.6x² | 1.75x | 767x |
| TGF-β | 484.2x | 1553.5x | 0.05x |
| IL-10 | 170.5x | 100x | 67.2x |
| TNF-α | 0.01x | 7x | 90x |

¹IVIG was added to the IVIG tx cultures on day 7, 2 days prior to addition of LPS.
²Relative gene expression was measured using the 2\hat{ }(−ΔCt) method. All data obtained from the different treatments for the genes of interest were first normalized to their respective GAPDH Ct levels prior to comparison to control BMDC.

IVIG Treated Sensor Cells Protects Mice from Lethal Encephalomyelitis.

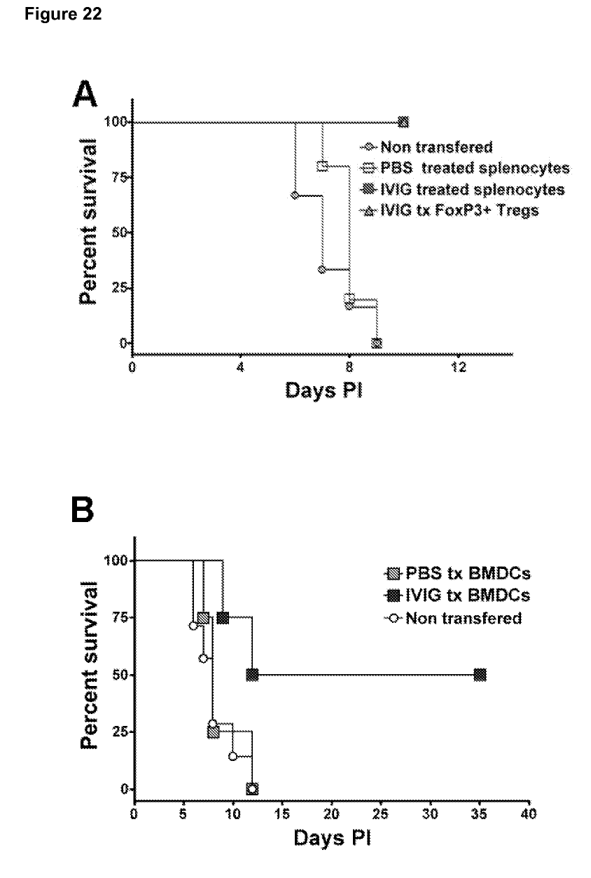

The model demonstrates that IVIG primed s-cells have the ability to protect mice from death. Splenocytes prepared from naïve 129 mice treated for 2 h with either PBS or IVIG were transferred into recipients which were then challenged with HSV 17+ by corneal scarification. IVIG treated splenocytes conferred complete protection whereas recipients of PBS primed splenocytes or non-transferred recipients all died within 7-10 or 6-8 days pi respectively (FIG. 22A). Recipients which received PBS primed splenocytes had florid periocular skin and eye disease, which was completely absent in recipients of IVIG primed splenocytes. These results show the S-cell, indicating a regulatory DC is present in spleen; consistent with the observed accumulation of myeloid DCs in IVIG treated mice.

To determine if BMDCs could provide protection similar to in vivo IVIG primed splenocytes, 129 Rag BMDCs were treated with IVIG or PBS on day 7 of culture and 24 h later were transferred into wt 129 recipients that were then infected with HSV. 50% of IVIG primed DC recipients were protected from lethal HSE and the onset of death was delayed by 4-6 days relative to control mice for those mice that died, while all PBS primed DC recipients succumbed to HSE in a similar time frame to control non-transferred mice (FIG. 22B).

### Regulatory T-Cells (Tregs) Induced by IVIG Treatment Mediate Protection in Adoptively Transferred Mice.

IVIG induces a dramatic expansion of Tregs in HSV infected wt 129 mice. Their role in suppressing inflammation was identified. Purified Tregs sorted from spleens of HSV immunized (ip inoculation of HSV 17+) IVIG treated 129-FoxP3-GFP mice on day 5 pi were adoptively transferred into wt 129 mice that were infected with HSV 2 h later and monitored for signs of HSE. As shown in FIG. 2A all recipients of FoxP3+ Tregs were completely protected compared to recipients of control untreated splenocytes from naive mice that contain nTregs. Moreover, although Tregs expand about 3 fold in HSV infected wt 129 mice they fail to protect.

129-Rag Mice Are Not Protected by Adoptive Transfer of Naive CD4+ T cells.

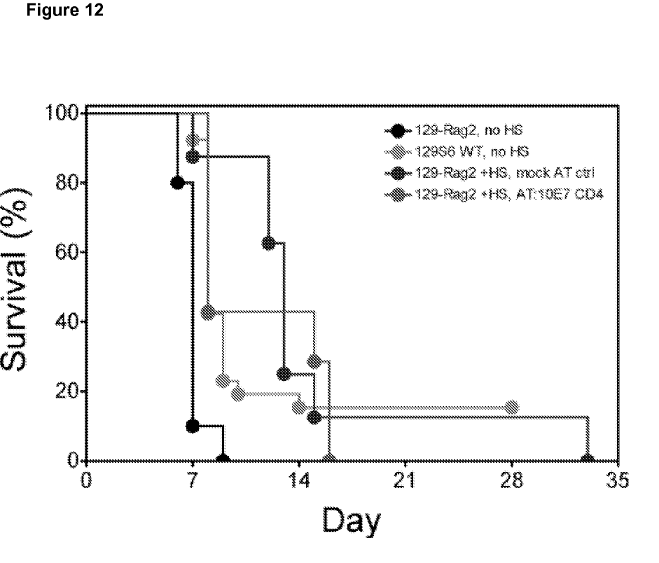

Naive CD4+ T cells purified by positive selection from dissociated 129 spleen cell suspensions by positive selection using an AutoMacs instrument (Miltenyi Biotec) were >96% pure as determined by staining for CD4. Deeply anesthetized 129-Rag mice (5 per group) were infused with 1×10⁷ CD4+ T cells by tail vein injection and immediately inoculated with HSV by corneal scarification. Mice were passively immunized with 500 ml HS 24 h later or given PBS as control (it is difficult to obtain human serum free of HSV specific antibody) and monitored daily. Adoptive transfer of CD4+ T cells together with passive immunization did not protect 129-Rag mice against fatal HSE (FIG. 12). This indicates that CD4+ T cells are not the ‘P-cell’ type missing in Rag mice that is responsible for IVIG dependent protection in WT 129 mice.

### Microarray Analysis for Cytokine Gene Expression in HSV Infected Brainstem.

RT2 real-time PCR Inflammation array (Innate response/TLR genes) was used to monitor gene expression in the brainstem of uninfected and infected 129 and B6 mice at early times after corneal inoculation, day 3 which is the earliest time HSV is detected in the brainstem and day 4 PI when replicating HSV can easily be detected. Processing of the arrays was done as per instructions in the kit. A summary of this analysis is presented in Table 4 and serves to validate the expression profiling experiments to examine whole Tgs and pooled Tg neurons for expression of innate response genes. It is notable that inflammation related genes are differentially induced by HSV infection in the brainstem of B6 and 129 mice consistent with the idea that B6 and 129 innate responses are different. Furthermore, Irf1 is upregulated significantly in 129 infected brainstem at day PI consist with the DNA oligo microarrays results presented in our recent paper on CXCR3 signaling in HSV infection (67). The RT2 real time array platform to be highly sensitive and user friendly with inter sample variation based on expression of control genes being <3%, which lends a high degree of confidence to results from comparative analysis of samples from mice treated or not treated with IVIG.

TABLE 4
Gene expression analysis of brainstem samples from 129S6
and C57BL/6 mice on day 3 and 4 post-infection.

| | | |
|---|---|---|
| Gene name | Day 3 | Day 4 |

A. Genes induced in BS by HSV infe [symbol] n
129S6 mice (vs MOCK)

| | | |
|---|---|---|
| Ccl2 | nd | 124.9 |
| Irf1 | nd | 16.8 |
| Ptgs2 | nd | 12.2 |
| Eif2ak2 | nd | 3.9 |
| Ly86 | nd | 3.2 |

C57BL/6 mice (vs MOCK)

| | | |
|---|---|---|
| Ccl2 | nd | 52.0 |
| Ifng | nd | 19.0 |
| Clec4e | nd | 13.0 |
| Il1b | nd | 8.1 |
| Tlr6 | nd | 7.2 |
| Ticam2 | 3.0 | 7.0 |
| Il6 | nd | 6.5 |
| Tnf | nd | 6.4 |
| Irf1 | nd | 5.6 |
| Il2 | nd | 5.2 |

B. Mouse strain comparison
129S6 greater than C57BL/6 mice

| | | |
|---|---|---|
| Hmgb1 | 3.0 | 3.0 |
| Ticam2 | 3.0 | nd |
| Pglyrp1 | 3.1 | nd |
| Ptgs2 | 3.1 | (3.4) |
| Hspa1a | 4.8 | nd |

C57BL/6 greater than 129S6 mice

| | | |
|---|---|---|
| Tnf | 7.1 | 4.7 |
| Il1b | 4.6 | 4.6 |
| Csf3 | 4.1 | 8.5 |
| Nfkb1 | 3.2 | nd |
| Ccl2 | 3.1 | 5.4 |
| Ifnb1 | nd | 6.3 |
| Cd14 | nd | 6.0 |
| Il6 | nd | 5.2 |

nd = not different (<3.0)
A. Expression levels compared to uninfected. B. Expression levels comparison between strains. Genes that are induced at least 3-fold (day 3) or 5-fold (day 4) are shown is this composite data from seven experiments using Superarrays' RT2 real-time inflammation array. [symbol]
indicates data missing or illegible when filed

Use of LCM to isolate HSV infected Tg neurons for microarray analysis; infection with a HSV strain expressing a marker gene (e.g. LacZ or EGFP) will facilitate identification of infected neurons.

### Expression of Inflammation Related Genes in Brain Stem (BS) and Trigeminal Ganglia (Tg)

HSV infected 129-Rag mice died rapidly starting as early as day 6 pi. A marked increase in inflammatory macrophages/microglia has evident in the BS at day 5 pi in untreated mice but was dramatically reduced in IVIG treated mice. Expression of the chemokines, MIG and IP10, is highly upregulated in the Tg by day 4 pi and signaling is detrimental in 129 compared to B6 mice (66). Moreover, protection against HSE declined sharply when IVIG was administered later than 24 h pi. This suggested that critical changes involving induction of an immunoregulatory cell (S-cell) occurred rapidly after infusing IVIG at 24 h pi and these cells then modulated inflammatory responses of effector innate cells (e.g., macrophages and neutrophils) to achieve virus control without bystander tissue damage.

To gain insight into the nature of these changes, expression of inflammation related genes will be profiled in BS, Tg and draining cervical LN/spleens of untreated and IVIG treated HSV infected and mock infected 129 wt and Rag mice on day 2 pi, one day before HSV first appears in the BS on day 3 pi, and day 6 pi when robust inflammation is observed in the BS. Mock infected mice will serve as controls for induction of inflammatory responses resulting from the mechanical stress of HSV corneal inoculation that has the potential to affect Tg responses because sensory nerve endings enervate the corneal epithelium (3 mice/group, 24 mice total). Comparison of inflammatory responses in wt versus Rag mice at these early time points preceding maturation of adaptive responses should reveal any modulatory effects of T cells on innate responses. Naïve T cells were recently reported to temper early innate responses in an antigen independent manner and subsequent studies showed that neonates lacking a fully mature adaptive system develop uncontrolled proinflammatory responses after LPS challenge or viral infection (26, 128).

Individual BS and Tg from 3 mice/group (129 and 129-Rag) were profiled. Expression profiles for TLRs, cytokines, chemokines and their receptors were compared in the Tg and BS of untreated or IVIG treated HSV infected or mock infected 129 and 129-Rag mice at day 2 and 6 pi. Expression profiles of inflammation related genes obtained in secondary lymphoid organs (e.g., dLN and spleen) were compared to profiles from neural tissues (BS and Tg) in 129 wt and Rag mice to provide information on (i) IVIG induced differences in cytokine/chemokine expression in lymphoid organs and neural tissues, (ii) the inflammatory profile in untreated versus IVIG treated wt and Rag mice and (iii) differences in expression of these molecules in the presence (i.e. wt 129) and absence (i.e. 129-Rag) of adaptive cells. The activation state of DCs and macrophages in the dLN, spleen and BS at day 2 and 6 pi with expression profiles for cytokines, chemokines and TLRs are correlated; DCs were not present in the BS day 6 pi but IVIG induced accumulation of pDCs in BS later in infection.

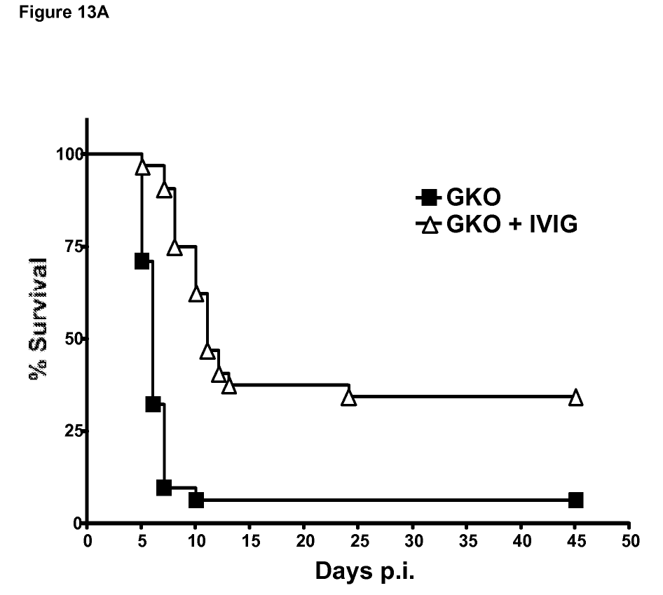

Significant quantitative and qualitative changes in expression of chemokines, cytokines and their associated receptors are expected in comparison of HSV infected IVIG and PBS treated 129 wt and Rag mice. This will reflect a shift in innate monocyte populations from a classically activated proinflammatory Th1-like (M1) state to an alternatively activated anti-inflammatory Th2-like (M2) immunoregulatory state (71). Changes in expression profiles for a broad array of molecules including membrane receptors, chemokines and cytokines and associated receptors are expected for effector cells (macrophages and neutrophils) in BS and Tg, as well as for APCs in dLN and spleen as depicted in FIG. 13 for macrophages.

### Imaging IVIG Biodistribution and HSV Spread.

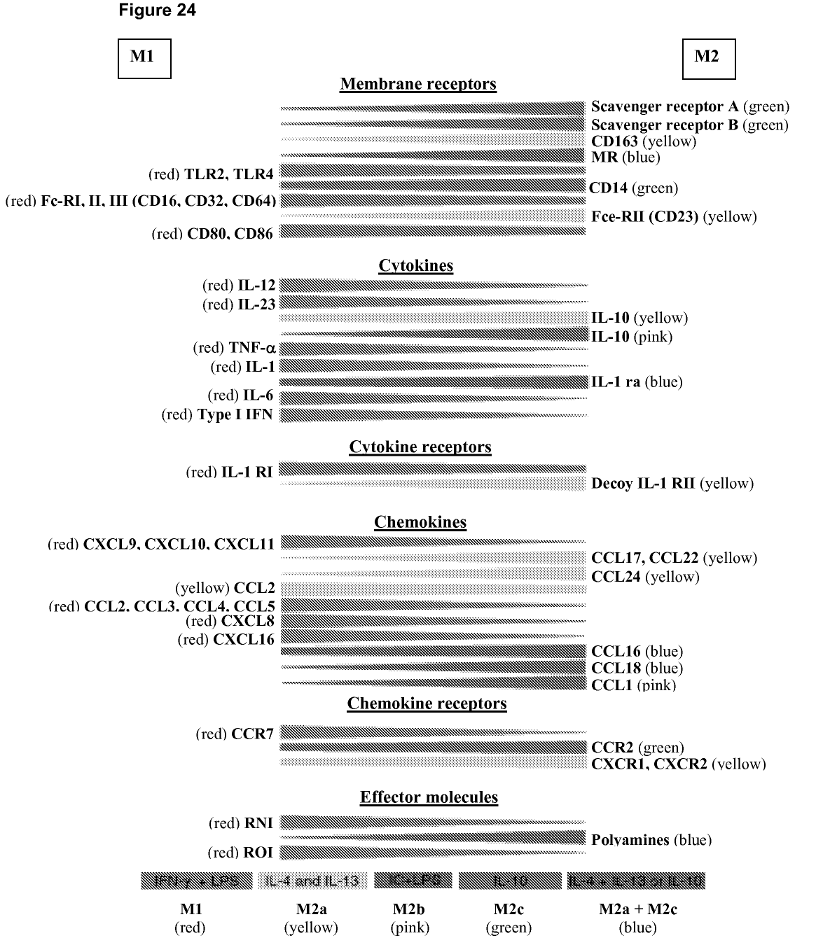

Purified m-IVIG labeled with ⁶⁴Cu DOTA was imaged using serial small animal positron emission tomography (PET) to monitor the distribution of human IgG in mice infected with HSV. Labeling and microPET imaging was done in the small Animal Imaging Core. The most significant finding was that IgG did not enter the brain at day 4 pi and essentially distributed in the vasculature with no difference between infected and uninfected mice as determined by counting radioactivity in different tissues after imaging the mice, (FIG. 24). Thus, IVIG immunomodulatory effects occurred peripherally since differences in mononuclear cell infiltration in the BS were already apparent by day 4 pi. Imaging was done at day 4 and 8 pi to determine whether later in infection IgG enters the BS of IVIG treated mice; 4 mice/group, 8 mice total. Because the half-life of ⁶⁴Cu for imaging is 48 h, mice were treated with unlabeled IVIG at 24 h pi and infused with tracer amounts (≦10 mg) of ⁶⁴Cu-IgG on day 4 and day 8 pi for imaging. One caveat is that competition with unlabeled IVIG can diminish the signal so as to preclude unambiguous assignment of IVIG distribution. This being the case, mice infused with IVIG at 24 h pi were perfused prior to sacrifice and processing of various tissues (e.g., BS and brain) on day 4 and 8 pi for detection of human IgG by immunostaining of thin sections or ELISA assay on BS homogenates. Data on biodistribution of IVIG at different times pi aided in elucidating the anti-inflammatory mechanism and reveal the potential for IVIG interaction with infiltrating mononuclear cells in the CNS. Although, IVIG preserves the integrity of the BBB at late times post infection, IgG will gain entry to the CNS.

There is a potential for differences in the spread of HSV in different mouse strains and in immunodeficient compared to wt mice. To investigate this possibility, mice were infected with a HSV strain expressing firefly luciferase, KOSDlux (63) and spread were monitored in live untreated and IVIG treated wt 129 and 129/B6-Rag mice using the Xenogen bioluminescence imaging (BLI) system available in the small Animal Imaging Core. Mice injected with the luciferase substrate, D-luciferin were repetitively imaged on day 2, 4, 6 and 8 pi as previously described; 4 mice/group, 12 total) (63). The substrate permeates cell membranes and the BBB thereby allowing BLI of HSV infection in any anatomical site with high sensitivity because of the low background signal. Differences in spread between 129 wt and 129-Rag strains are expected. Enhanced spread in 129 compared to B6 mice can also account for the widespread activation of microglia in 129 compared to B6 mice that likely contributes to the observed hyperinflammatory responses in the CNS of 129 mice.

### Construction of a ‘129-Empty’ Mouse (IL-7R^{-/-}Kit^{W41/W41}).

B6-E mice homozygous for IL-7R^{-/-} and Kit^{w41/w41} mutations are blocked at a very early stage of hematopoiesis before TCR rearrangement hence, they lack B cells, ab and gd T cells, whereas the NK cell compartment is normal. IL-7Ra^{-/-} and Kit^{w41/w41} mice were generated on the 129 background crossed them to generate the 129-E mice strain. Derivation of 129-IL-7R^{-/-} mice was accomplished by using the original ES clone to generate male founder mice that were crossed to 129 females as was done previously to isolate the Ifng null mutation in the 129 strain background (11). A targeting vector harboring the Kit^{w41/w41} mutation was linearized and electroporated into W9.5 ES cells (129S1/SvImJ) and clones having the correct Kit^{w41} mutation were identified by PCR analysis and DNA sequencing. Two correct clones were selected for injection into C57BL/6 blastocysts and founders were crossed to 129 females. Heterozygous offspring showing germline transmission were crossed to a universal 129S1/SvImJ Cre deleter strain (121) to obtain heterozygous male and female mice retaining the Kitw41 mutation but deleted for Neo; these were crossed to isolate the 129-Kit^{w41/w41} strain, referred to as 129-Kit^{W41/EC}. The 129.IL-7R^{-/-} and 129.Kit^{w41/w41.EC} strains have been crossed to generate the ‘129-empty’ (IL-7R^{-/-} Kit^{w41/w41}) strain and a breeding colony has been established and shown to breed well.

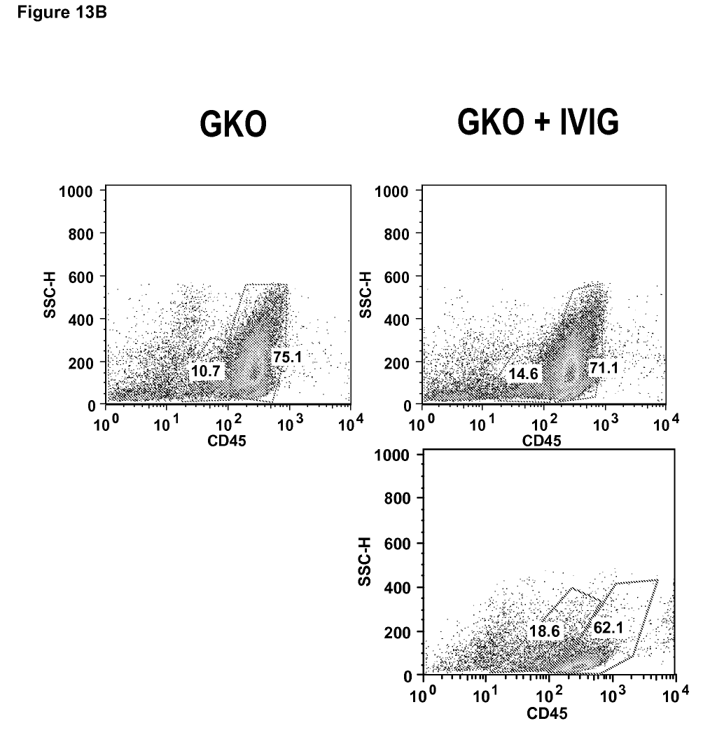

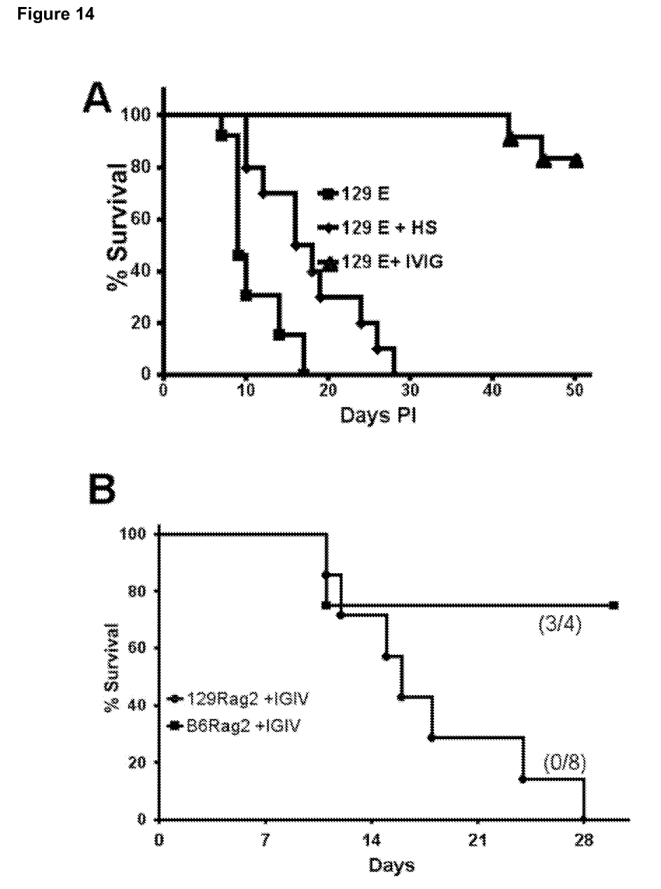

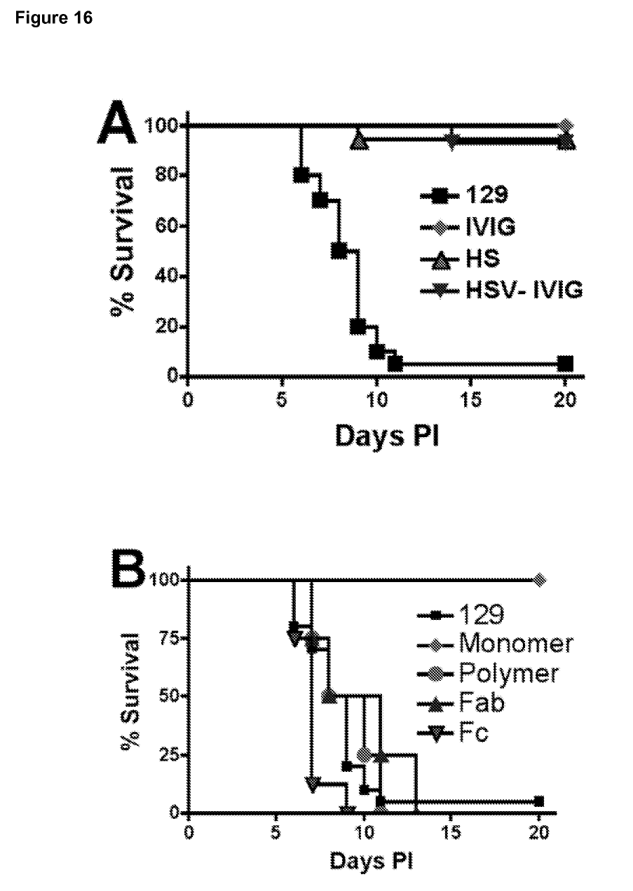

B6 mice, including the lymphopenic Rag and E strains are significantly more resistant to HSE than the corresponding 129 strains. Fatal HSE in 129 mice results from destructive inflammatory responses in the brainstem, whereas death of B6 is attributable to virus cytopathology. B6-E mice are significantly more resistant to HSE than B6-Rag mice; approximately 50% survive and harbor latent infections in their Tg whereas all B6-Rag mice die. 129-E mice are also more resistant than 129-Rag mice. What distinguishes E mice from Rag mice is that they lack mast cells, which implicates mast cells in the increased susceptibility of Rag mice. Treatment with pooled human serum (HS) results in long-term survival of >80% of HSV infected B6-Rag and B6-E mice. Wild type 129 and 129-Rag mice are both highly susceptible to fatal HSE but while HS treatment protected 100% of WT mice, 129-Rag mice were not protected despite a regimen of repeated dosing, including with co-administrated ACV to block HSV replication. Results show that while a single dose of IVIG given 24 h PI protected >80% of 129-E long-term, 129-Rag mice were not protected (FIG. 14A) and surprisingly HS protected neither 129-E nor 129-Rag mice although it did protect B6-Rag mice; this is the first observed significant difference between HS and IVIG (FIG. 14) although slight differences have also been seen with WT 129 mice (FIG. 16). However, IVIG is used exclusively since it is a better-defined and more rigorously controlled product than pooled human serum which is not intended for use in humans. These findings warrant further investigation in light of recent reports that mast cells, being strategically located at mucosal barriers and in close proximity to nerve tracts and blood vessels, contribute to innate defenses against bacterial and viral pathogens and are also involved in induction of adaptive responses (34, 40, 73, 83).

Careful consideration of our data and the biology of HSV led us to propose a model involving two mechanisms for IVIG mediated protection against fatal HSE in 129 mice. One mechanism involves modulation of the inflammatory response to minimize bystander damage to the CNS and the second mechanism involves suppression of HSV replication in neurons. Both mechanisms are operative in the susceptible 129 strain mice including the Rag/E strains, while only the second mechanism is required for protection of B6-Rag/E mice. Compared to WT, protection is short lived in 129-Rag mice as deleterious inflammatory effector macrophages reappear in the brainstem accompanied by reemergence of replicating HSV about 10-12 days after cessation of IVIG treatment (including±ACV) and all mice eventually die (FIG. 10). Since, HSV spreads via retrograde intra-axonal transport, early and prolonged treatment of Rag mice with IVIG±ACV effectively confines HSV exclusively to neurons. Therefore, IVIG mediated protection of B6-Rag mice but not 129-Rag mice reveals a fundamental difference in the capacity of neurons from these two strains of mice to restrict HSV replication in response to IVIG treatment. Evidence supporting the concept of neuronal restriction of HSV replication has been reported for wild type and immunodeficient SCID mice (70, 125, 134). HSV infected B6-Rag mice that have unremarkable inflammatory responses in the brainstem, die because of failure to control replicating virus that eventually spreads and destroys critical neurons consistent with the observed slower kinetics of mortality. Long-term survival of B6-Rag/E mice treated 24 h PI with IVIG reveals the second mechanism of IVIG action, namely suppression of HSV replication in neurons.

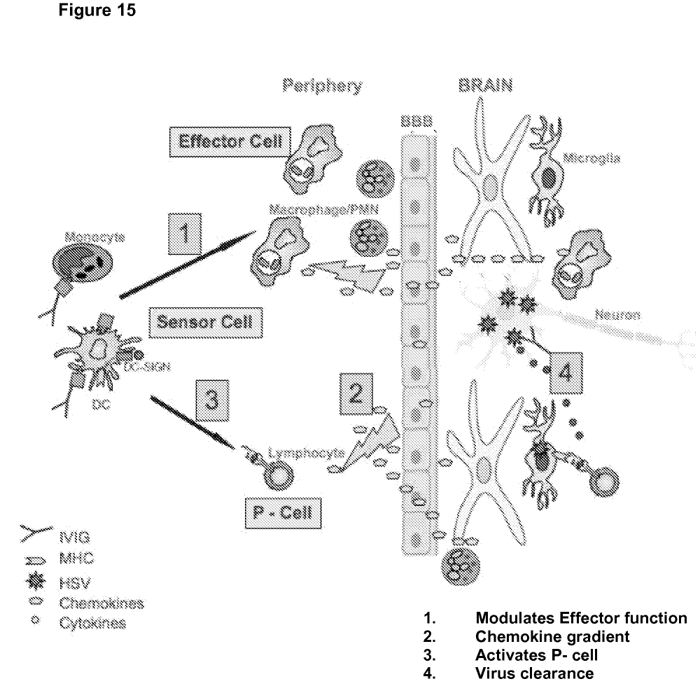

According to our model, illustrated in FIG. 15 the ‘sensor cell’ responding to IVIG, specifically sialylated IgG, acts to modulate the activation state of effector macrophages (and other innate effector cells such as neutrophils) rendering them less aggressive (1); this may involve diffusible mediators or direct cell interaction and it may result in reduced infiltration into the brain. Modulation of the S-cell, which is very likely an activated APC (DC or macrophage), does not require antigen specificity. Activated effector macrophages infiltrate the Tg and traverse the BBB to enter the brainstem where they are found in close contact with neurons (2). In WT 129 mice, the ‘P-cell’ activated by the ‘S-cell’ enters the Tg and brainstem in response to chemokine signals produced by infiltrating macrophages and or endogenous innate cells in the brain (3). Activated macrophages, P-cells and possibly other innate cells such as neutrophils and microglia, collaborate with HSV specific IgG bound to infected neurons or surrounding glial cells to initiate signaling events that lead to suppression of HSV replication in neurons (4). gd T cells are favored as the P-cell candidate. Suppression of HSV replication by IVIG given 24 h PI is transient in 129-Rag mice lacking P-cells and resumption of replication reinitiates pathogenic inflammatory responses culminating in death. In contrast, a single IVIG treatment at 24 h protects B6-Rag/E mice lacking P-cells, reflects fundamental differences in the ability of B6 and 129 neurons to restrict HSV replication. 129-E mice lacking both P-cells and mast cells respond to IVIG like B6-Rag/E mice, which implicates a role for mast cells. In this type of IVIG protection the anti-inflammatory mechanism does not require HSV specific antibodies and suppression of viral replication in WT 129 mice is enforced by adaptive immune responses. In contrast in immunodeficient mice, HSV specific IgG is required for suppression of replication and this may require antigen specificity for HSV. A single IVIG treatment fails to promote long-term survival of 129-Rag mice because suppression of both inflammation and HSV replication is transient due to the absence of the ‘P-cell’ that is responsible for long-term protection of IVIG treated WT 129 mice. Assuming resumption of inflammation in 129-Rag mice results from renewed HSV replication this would indicate that the ‘P-cell’ functions to enforce long-term repression of HSV replication and thereby, resolution of the inflammatory response which would promote long-term survival. The following observations provide support for our model: (1) macrophages are the predominant infiltrating cell in the Tg and brainstem where they control HSV replication (54, 69); (2) gd T cells that recognize HSV antigen independently of MHC restriction and macrophages cross-regulate their accumulation in the infected Tg and cooperate to control HSV replication (54, 65, 114). (3), although macrophages control HSV replicate in infected 129 mice they contribute to bystander tissue damage causing fatal HSE compared to protective effects in B6 mice (69). (4), T cell responses occur too late to play a major role in controlling HSV replication in the periphery and spread to the nervous system (12, 58, 126). Because the anti-inflammatory activity of IVIG is antigen independent, it is possible to glycoengineer IgG to exert potent anti-inflammatory activity that will have utility for treating HSE, HSK and other neuroinflammatory disorders (64).

### Anti-Inflammatory Effects of IVIG in Encephalitis

CNS inflammatory responses are characterized to HSV and to determine the effects of treatment with IVIG and its deglycosylated and desialylated derivatives on these responses, on survival and on viral load in the CNS. Integrity of the blood brain barrier (BBB), spread of HSV in the CNS and development of inflammatory lesions in the brainstem of non-treated and IVIG treated mice can be monitored and additionally compare biodistribution of IVIG in mock and HSV infected WT and Rag/E mice.

To deduce the anti-inflammatory mechanism responsible for IVIG protection against fatal encephalitis it is essential to have a comprehensive view of inflammatory responses in the brainstem following HSV inoculation on the cornea.

### Characterizing the Active Component of IVIG and the Role of IgG Glycosylation.

Monomeric IVIG (mIVIG) but not aggregated IVIG, the F(ab)2 or the Fc domain fractions are protective in the HSE model (FIG. 16B). Most importantly, FIG. 16A shows that IVIG lacking neutralizing antibodies (titer<1:2) retains protective efficacy for WT 129 mice but shows impaired protection of Rag/E strains. Glycosylation of asapargine 297 in the Fc domain is essential for maintaining a functional Fc structure and for binding to all FcgRs (50). Humans and mice with autoimmune diseases such as rheumatoid arthritis have altered IgG glycoslyation patterns with increased levels of antibodies lacking terminal sialic acid and galactose residues. Recently, Ravetch et al (50) showed convincingly that the FcgRIIB dependent anti-inflammatory activity of IVIG resulted exclusively from sialylation of Asp 297 in the Fc domain of a minor fraction of the total IgG molecules. Sialylated anti-inflammatory IgGs have reduced affinity for FcRs, indicating the existence of a novel IVIG receptor distinct from FcgRs (20).

The importance of glycosylation and sialylation of IgG Asp297 in the Fc domain for protection of 129 WT mice can be assessed by evaluating the protective capacity of deglycosylated and desialylated IVIG, focusing specifically on anti-inflammatory effects for desialylated IVIG. IVIG is treated in vitro with Peptide:N-Glycosidase F (PNFGaseF; New England Biolabs) or with recombinant a2,3/a2,6 neuraminidase (New England Biolabs) to deglycosylate or desialyate IgG, respectively (50). Deglycosylated and desialylated IVIG purified by HPLC to isolate the monomeric fraction can be tested for protection of WT 129 mice infected with HSV. Deglycosylation could abrogate both IVIG mechanisms and hence protection confirming importance of IgG Fc domain. IgGs enriched for sialylation on Asp297 can be isolated from IVIG using lectin affinity column purification as described by Kaneko et al. (50). No effects on the structural integrity or the monomeric composition of IVIG were seen after desialylation, hence IVIG mediated suppression of HSV replication can be intact. Dose ranging studies can be done to determine the minimal dose of sialylated IVIG (s-IVIG) that protects 100% of WT 129 mice from fatal HSE, with the expectation that a dose ˜10 fold lower compared to unfractionated IVIG can be protective. The minimal protective dose of whole IVIG is 1.2 mg/25 g mouse; doses of 1.2, 0.6, 0.3 and 0.15 mg can be tested (6 mice/group and 6 mice treated with PBS can serve as controls; 24 mice in all). The minimal protective dose of s-IGIV for WT 129 mice did not protect B6-Rag/E or 129-Rag/E mice, but potent anti-inflammatory activity can be evident in the 129-Rag/E strains (8 mice per strain; 32 mice for 4 strains). In the ITP model IVIG effects are strictly dependent on FcgRIIB expression and IVIG increased the proportion of FcgRIIB+ monocytes in spleen and bone marrow compared to PBS treated mice. While the majority of macrophages and neutrophils isolated from spleens of 129 and IVIG treated 129 mice were FcgR2B+, the cells isolated from IVIG treated spleens had higher MFI (Table 4). Moreover, there were many more macrophages recovered from the spleens of the IVIG treated mice. The FcgR1 profile revealed dramatic differences in both macrophages and neutrophils isolated from spleens of control mice with much fewer cells being positive for FcgR1 compared to IVIG treated splenocytes (Table 4). Thus the ratio of FcgR2b to FcgR1 was very different in macrophages and neutrophils isolated from the 2 groups of mice. s-IVIG effects on accumulation of monocytes expressing FcgRIIB in the spleen and bone marrow can be examined by flow cytometry in HSV infected 129 mice at day 4 PI, i.e. 48 after sIVIG treatment.

### IVIG Bioactivity of One IgG Subclass

IgG subclasses can be purified to determine whether the anti-inflammatory activity of monomeric IVIG resides within one of the four human IgG subclass (IgG1-IgG4) that bind with varying affinity and specificity to the different FcgR receptors (90). Affinity purification columns can be made by coupling monoclonal antibodies to human IgG2 and IgG4 (Calbiochem) to Ultralink Biosupport medium (Pierce Biotechnology) following instructions in the kit. These IgG subclass antibodies show high specificity and only minimal cross-reactivity and have been used to purify human IgG subclasses; hence this published procedure can be used (119). Briefly, monomeric IVIG can be run through the anti-IgG4 column to isolate IgG4 and deplete this subclass from m-IVIG. The effluent is loaded onto a protein A affinity column to isolate IgG3, followed by isolation of IgG2 on an affinity column. The final effluent can contain IgG1 with an expected purity of >95% (119).

Western blotting can be done to assess purity of the isolated subclasses. The relative proportion of each subclass in m-IVIG can be determined by ELISA assay using anti-human subclass antibodies (Calbiochem). The purified subclasses can be tested for protection against fatal HSE in WT 129 at doses proportional to their representation in m-IVIG (5 mice/group, with PBS as control). HSV specific antibodies reactive with protein lysates from infected Hela cells can be measured for each subclass by ELISA assay. Should none of the subclasses provide complete protection, the subclasses with anti-inflammatory activity and inhibitory activity for HSV replication can be identified—mice sacrificed at day 6 PI can be screened for brainstem infiltrates and reduced HSV titers compared to control PBS treated mice. In combination these two subclasses can provide protection.

### Requirement for Antigenic Specificity.

Although IVIG devoid of HSV neutralizing antibodies mediates protection of wild type 129 mice (FIG. 16), it fails to protect either B6-Rag or 129-Rag mice long-term though it did extend survival of B6-Rag mice (data not shown). Since, protection of B6-Rags depends solely on IVIG promoting suppression of HSV replication, this implies that adsorbed IVIG has lost capacity to suppress replication while retaining anti-inflammatory activity. Suppression of HSV replication is observed in WT mice that nonetheless die because of unabated CNS inflammatory responses. Anti-inflammatory sialylated IgGs protects WT 129 mice despite lacking antigenic specificity for HSV.

The finding that both a non-neutralizing and a neutralizing mAb protected WT 129 but not B6-Rag mice from death implies an anti-inflammatory mechanism is involved. HSV infected B6-Rag, 129-Rag and 129 WT mice, treated at 24 h with IVIG, adsorbed IVIG or each of the mAbs can be sacrificed at day 6 PI and virus titers and inflammatory infiltrates can be assessed in the brainstem. The relative effect on anti-inflammatory but not antiviral activity can be determined in these antibody preparations. The glycosylation profile of PAGE purified mAbs can be determined by analysis of tryptic peptides by liquid chromatography in combination with tandem mass spectroscopy (MS/MS). MS analysis is method of choice for glycosylation profiling, including sialyation of antibodies (50, 132). The expectation is that the mAbs can be sialylated.

A panel of baculoviruses expressing seven major HSV glycoproteins can be used to screen mIVIG to determine the dominant antigenic specificities present. IVIG adsorbed with individual baculovirus extracts can be tested for protection and suppression of HSV replication in 129- and B6-Rag mice in attempt to define the antigenic specificity involved in suppression of HSV replication. Involvement of more than one antigenic specificity was reflected by only partial protection and impaired suppression of virus replication. The protective capacity of IVIG adsorbed repeatedly with HSV infected cells to remove HSV specific antibodies (as determined by ELISA) was compared to that of whole IVIG in HSV infected B6-Rag and 129-Rag mice with the expectation that impaired suppression of replication, but not inflammation, can correlate with lack of protection against mortality.

### Imaging IVIG Biodistribution and HSV Spread.

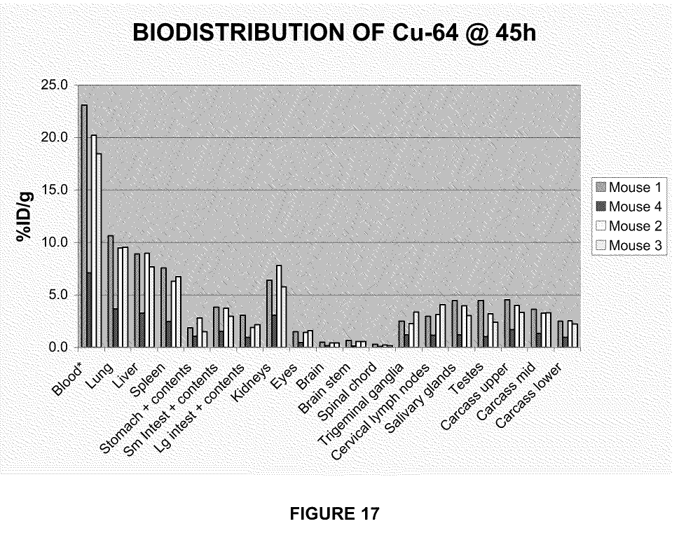

Imaging purified monomeric IVIG can be labeled with ⁶⁴Cu DOTA and using serial small animal positron emission tomography (PET) to monitor the distribution of the human IgG in mice infected with HSV. The most significant finding was that IgG does not enter the brain at day 4 PI and essentially distributes in the vasculature with no difference between infected and uninfected mice (FIGS. 2 and 17). Thus, IVIG immunomodulatory effects occur peripherally since differences in mononuclear cell infiltration in the brainstem are already apparent by day 4 PI. Differences in biodistribution of labeled ⁶⁴Cu-labeled IVIG can be determined in HSV infected mice not treated or treated with IVIG at 24 h PI. Imaging can be done at day 4 and at day 8 PI to determine whether later in infection IgG enters the brainstem of IVIG treated mice. Because the half-life of ⁶⁴Cu for imaging is 48 h, mice can be treated with unlabeled IVIG at 24 h PI and infused with tracer amounts (≦10 mg) of ⁶⁴Cu-IgG on day 4 and day 8 PI for imaging. One caveat is that competition with unlabeled IVIG may diminish the signal so as to preclude unambiguous assignment of IVIG distribution. This being the case, mice infused with IVIG at 24 h PI can be perfused prior to sacrifice and processing of various tissues (e.g., brainstem and brain) on day 4 and 8 PI for detection of human IgG by immunostaining of thin sections or ELISA assay on BS homogenates. Data on biodistribution of IVIG at different times PI can aid in elucidating the mechanism whereby IVIG suppresses HSV replication in neurons.

There is a potential for differences in spread of HSV in different strain backgrounds and in immunodeficient compared to WT mice. Mice can be infected with a HSV strain expressing firefly luciferase (KOSDlux, (66)) and spread can be monitored in live untreated and IVIG treated WT 129 and 129/B6-Rag/E mice using the Xenogen bioluminescence imaging (BLI) system available in the small Animal Imaging Core. Mice injected with the luciferase substrate, D-luciferin can be repetitively imaged on day 2, 4, 6 and 8 PI as has been described (66). The substrate permeates cell membranes and the BBB thereby allowing BLI of HSV infection in any anatomical site with high sensitivity because of the low background signal. Differences in spread between 129-Rag/E compared B6-Rag/E strains can be informative, especially if spread is more extensive in 129-Rag/E compared to B6-Rag/E strains as this can reinforce the notion of superior innate responses capable of restricting HSV existing in B6 mice.

### Expression of Inflammation Related Genes in BS, Spleen and dLN.

HSV infected 129-Rag mice die rapidly beginning day 6 PI (FIG. 2). A marked increase in activated inflammatory cells (macrophages/microglia) is evident in the BS at day 5 PI in untreated mice but is dramatically reduced in IVIG treated mice. Expression of the CXCR3 chemokines, MIG and IP10, is highly upregulated in the Tg by day 4 PI and we have shown that CXCR3 signaling is detrimental in 129 compared to B6 mice (67). Moreover, protection against HSE declines sharply when IVIG is administered later than 24 h PI. This indicates that critical changes involving induction of immunoregulatory cells occurs rapidly after giving IVIG at 24 h PI and these cells then modulate inflammatory responses of effector innate cells (e.g., macrophages and neutrophils) to achieve virus control without bystander tissue damage. To gain insight into the nature of these changes expression of inflammation related genes can be profiled in BS of untreated and IVIG treated HSV infected mice on day 2 PI, one day before HSV first appears in the BS on day 3 PI and on day 5 PI, when robust inflammation is observed in the brainstem. Individual BS from 3 mice/group (129, 129-Rag/E and B6-Rag/E) can be profiled.

Expression profiles for TLRs, cytokines, chemokines and their receptors can be compared in the Tg, BS and brain of untreated or IVIG treated 129, 129-E/Rag and B6-E/Rag mice at day 2 and 5 PI (1 and 5 days after IVIG administration, respectively). Comparative expression profiles for these immune molecules in secondary lymphoid organs (e.g., dLN, spleen) versus CNS compartments in WT 129 versus Rag/E strains can provide information on (i), IVIG induced differences in cytokine/chemokine expression in secondary lymphoid organs and/or CNS tissues (ii) differences in expression of these molecules in the presence (ie WT 129) and absence (ie 129-Rag) of the ‘p-cell’ and both the ‘p-cell and mast cells (129-E) (iii), the inflammatory CNS profile in untreated versus IVIG treated mice. Additionally, we can correlate the activation state of DCs and macrophages determined in the dLN, spleen and brainstem at day 2 and 5 PI with expression profiles for cytokines and TLRs; DCs are not present in the brainstem day 6 PI but IVIG induces accumulation of pDCs in brainstem later in infection.

The RT²Profiler™ PCR Array System or Oligo GEArrays are pathway-focused Array platforms from SuperArray Bioscience Corporation (Frederick, Md., USA) that provide a convenient accessible method for gene expression profiling requiring only a real time PCR instrument or laser scanner for reading slide oligo arrays. Both systems combine high sensitivity and reliability in kit form and require only small amounts of total RNA ranging from 5-100 ng. The RT²Profiler™ PCR Array System exploits the superior quantitative power of real-time RT-PCR that has been pre-optimized to allow detection of multiple genes in specific pathways to provide the profiling capability expected of a microarray. The RT²Profiler™ array system is the method of choice if the appropriate target genes are represented otherwise Oligo arrays can be used and results confirmed using the RT²Profiler™ PCR Array. cDNA prepared from RNA extracted from the left half BS including the trigeminal nerve entry root zone region can be used for expression profiling with PCR arrays for mouse chemokines and receptors (APMM-022), inflammatory cytokines and receptors (APMM011) and interferon and receptor (APMM064). Inflammatory lesions in HSV infected 129 mice were localized to the trigeminal nerve entry root zone region of the BS by MRI. Both of these array platforms are currently in use in the lab (Table 3).

### Blood Brain Barrier (BBB) Integrity and Generation of Lesions.

The vigorous inflammation observed in 129 WT mice depicted by the massive CD45^{high} infiltrates (FIG. 6) indicates that the BBB is compromised rapidly following entry of HSV into the BS. Neutrophils and macrophages are among the first cells to invade the CNS and can rapidly induce BBB destruction. By contrast, the reduced inflammation observed in IVIG treated mice indicates that BBB integrity is maintained thereby limiting excessive inflammation. To determine if BBB integrity is conserved following IVIG treatment, sodium fluorescien uptake assays can be conducted (100). Briefly, infected mice that received or did not receive IVIG 24 hours PI can be injected IP with a 10% solution of sodium fluorescien. After 10 minutes, anesthetized mice can be bled, perfused with PBS, and brain, brainstem and spinal cord can be analyzed for uptake of sodium fluorescein in comparison to steady state serum concentration. Groups of 4 mice either treated or not treated with IVIG can be used at 3 different time points; d4, 6 and 8 p.i. (n=8×3=24) and BBB permeability can be compared to sodium fluorescein uptake in naïve mice (n=4; total mice=24+4=28 129 WT and 28 RAG mice). These experiments should reveal when initial BBB breakdown occurs and to what extent protection is afforded by IVIG. To demonstrate contribution of macrophages and neutrophils to the BBB breakdown, mice (n=4) can be irradiated (900Gy) and sodium fluorescein uptake compared to that in non-irradiated mice at day 6 PI when sufficient BBB breakdown have occurred.

MRI imaging and histology revealed extensive lesions in the brainstem of 129 WT mice by day 6 p.i. Macrophages and granulocytes predominated in these lesions but T cells were sparse. Depletion of macrophages and Gr-1+ neutrophils during acute infection resulted in significantly enhanced survival confirming a pathogenic role for these cells in fatal HSE. To determine if IVIG mediated inhibition of deleterious macrophage responses can suppress development of lesions, paraffin embedded sections derived from the right half of brainstems from IVIG treated and untreated 129 WT and RAG mice at day 6 and 8 PI (n=3/group per time point=12 WT and 12 RAG) can be H&E stained and scored for lesions by a pathologist. The impact of IVIG on the number and size of lesions can be observed in both WT and RAG mice. These experiments can help determine the contribution of macrophages/Gr-1+ neutrophils to lesion development in WT and RAG mice and the effect of IVIG on CNS immune pathology.

### Identification of the Cell Type(s) Required for IVIG Mediated Protection Against Fatal HSE in 129-RAG/E Mice.

An adoptive transfer strategy can be used to determine which of B, T, gd-T or NKT cell types is the ‘p-cell(s)’ required for long-term protection of adoptively transferred 129-Rag mice infected with HSV and subsequently treated or not treated with IVIG. The capacity for P-cells treated in vitro with IVIG to mediate long-term protection in adoptively transferred infected 129-Rag mice not treated with IVIG can be assessed to differentiate the ‘P-cell’ from the ‘sensor cell’. The effect of depleting the protective cell (P-cell) prior to IVIG treatment on protection of 129 mice can be evaluated.

Fatal HSE in susceptible 129 mice results from hyper-inflammatory responses in the brainstem mediated primarily by macrophages and neutrophils. IVIG given at 24 h PI exerts potent anti-inflammatory effects that result in 100% survival with significant reduction in accumulation of activated inflammatory macrophages in the BS of treated 129 mice. Thus, IVIG protection involves immunomodulatory effects. However, the remarkable observation that IVIG fails to protect 129-Rag mice, even with repeated dosing including supplementation with ACV, indicates a more complex scenario. Unlike in WT mice, IVIG anti-inflammatory effects and suppression of HSV replication are transient in 129-Rag mice; with activated macrophages and replicating HSV reappearing in BS about day 10 PI after a single IVIG treatment at 24 h PI (Table 2 and FIG. 10A). Based on studies with 129 WT and 129/B6-Rag/E mice a model for IVIG mediated protection of 129 mice was derived that involves two components: (i), suppression of hyper-inflammatory responses induced by HSV infection and (ii), suppression of virus replication. Finding a significant reduction in HSV titers in brain and BS of IVIG treated compared to non-treated 129 WT and Rag/E mice supports this model, especially because new data showing that IVIG devoid of HSV specific neutralizing antibodies protects WT 129 mice as effectively as IVIG with neutralizing antibodies. It was unclear whether IVIG treated 129-Rag mice died from resumption of viral replication or inflammatory responses in the brainstem or a combination of both insults. If immune mechanisms dominate, treating Rag mice with ACV at the time inflammatory macrophages reappear would not be protective; therefore the opposite would be true if viral cytopathology were dominant. Treatment with IVIG is protective irrespective of which mechanism dominates. These predictions of the model were tested and preliminary results are consistent with death being caused by destructive inflammatory responses since IVIG but not ACV treatment administered at day 14 PI was protective (not shown). P-cell(s) by virtue of ensuring suppression of virus replication promote resolution of the inflammatory response in the brain and thereby long-term survival. Although unlikely, two or more cell types might be required for IVIG mediated protection of 129-Rag mice.

Adoptive Transfer of Purified B, CD4+, CD8+, gd-T and NK T Cells.

Cell populations of interest can purified by negative selection yielding an ‘untouched’ population of highly purified cells. Unwanted cells magnetically labeled with MACS MicroBeads (Miltenyi Biotec Inc., Auburn Calif.) are retained on a MACS column placed in a magnetic field while the unlabeled target cells are collected as a highly pure cell suspension in the flow through. Adoptive transfer of naïve spleen cells depleted for CD3 (T cells) or IgG (B cells) into 129-Rag that are subsequently infected and then treated at 24 h PI with IVIG can exclude the P-cell being either a T or B cell. Assuming T cells are implicated, transfer of spleen cells depleted with an anti ab TCR specific mAb can test the protective capacity of gd T cells, since CD4, CD8 and NKT cells would be depleted. Spleen cells depleted with mAbs specific for NK1.1 (removes NK and NKT) and CD4 can evaluate CD8 T cells; CD4 T cells were excluded as the p-cell Adoptive transfer of HSV infected IVIG treated 129-Rag mice has excluded a role for CD4+ T cells (FIG. 12). Separate purified populations (5×10⁶-1×10⁷ cells) can adoptively transferred into 129-Rag mice by tail vein injection. The efficacy of antibody mediated depletion can be assessed by flow cytometry of stained spleen cells. Transferred mice can be infected with HSV the next day by corneal scarification and either treated or not treated with IVIG 24 h PI. Mice can be monitored daily for survival. Prior reports (29, 82, 93) and arguments above indicate that the ‘P-cell’ can reside in the T cell (CD3+) population. In the unlikely event, more than one T cell type is involved, this is reflected by partial or no protection when only one T cell type is transferred is transferred. Transfer of all possible T cell pair combinations (CD4+CD8, CD4+gd and CD8+gd) can reveal whether two or all three T cell types mediate protection. Transfers can be done using 8-10 mice per group.

### Analysis of HSV Infected Adoptively Transferred Mice IGIV Treated or Not Treated.

Having identified the P-cell it was determined that (i), in vitro effects of IVIG on expression of FcgRs, cytokines and chemokine and their receptors in IVIG treated and untreated P cells as well as on effector macrophages (ii), the capacity of in vitro IVIG treated P-cells to protect adoptively transferred 129-Rag mice and the effect of P-cell depletion on IVIG mediated protection of WT 129 mice (iii), in vivo localization of IVIG treated compared to non-treated P-cells (iv), BS expression profiles for type I IFN response genes and inflammatory cytokine/chemokine genes are involved in restricting HSV lytic gene expression in neurons in the presence and absence of IVIG.

### (i) In Vitro IVIG Effects on FcgR, Cytokine and Chemokine Expression in P-Cells.

P-cells can be incubated for ˜24 h with IVIG, washed thoroughly and then total RNA can be converted to cDNA which can be used to probe inflammatory cytokine/chemokine RT²Profiler™ PCR Arrays or Oligo arrays and used in real time PCR assays to monitor expression of FcgRs. Surface expression of receptors can be confirmed by flow cytometry for FcgR1 (anti CD64), FcgR2/3 (anti-CD16/32) and cytokine (e.g. IFNgR1/2) and chemokine receptors.

### (ii) In Vivo Protection by In Vitro IVIG Primed P-Cells.

WT-129 HSV infected mice adoptively transferred with in vitro IVIG primed P-cells (10⁶ washed cells given IV) or BSA primed P-cells (as control) at 24 h PI can be monitored for survival. Suppression of HSV replication by P-cells can be reflected by reduced titers virus titers in Tg, BS and brain. All mice can be infected with HSV 24 h prior to adoptive transfer of P-cells. This test serves to discriminate the P-cell from the S-cell since only in vitro primed S-cells should be competent to protect HSV infected WT 129 mice not treated with IVIG at 24 h PI. Infected 129-Rag mice treated with IVIG at 24 h PI can be adoptively transferred with P-cells either not primed or primed in vitro with IVIG at ˜36-40 h PI. If, both groups are protected it can imply that the S-cell determines the activity of P-cells but if neither group is protected this can reflect a requirement for both the P-cell and S-cell being present at the time of S-cell activation by IVIG. To confirm their protective role, 129 mice depleted of P-cells prior to HSV infection then treated with IVIG at 24 h PI can be monitored for survival and development of CNS inflammation; HSV titers in BS and brain can be determined. P-cells can be depleted by treating mice with mAb to a surface marker; anti-P-cell mAb given on day −2, −1 and +2 PI.

Mice are infected with HSV on day 0 and treated with IVIG 24 h PI; efficiency of depletion can be determined by flow cytometry analysis of spleen cells stained for P-cells. Additionally, depletion of P-cells in 129 mice on day 3, 6 and 8 PI can reveal whether their presence is required continuously or just at the critical interval (−24 through +48H PI) when treatment with IVIG is able to elicit protection. Survival and development of BS inflammation and suppression of HSV replication as determined by HSV BS titers at day 7 PI can be monitored. The capacity for IVIG primed P-cells to mediate protection in 129-Rag mice adoptively transferred at day 2 and 4 PI can be evaluated. The ability of in vitro primed P-cells to protect 129-Rag mice treated with IVIG from relapsing fatal HSE (which usually initiates ˜day 10-12 PI) when given at day 12 PI can be assessed. Effects on BS inflammation and HSV replication can be compared in P-cell recipient and non-recipient mice on day 15 PI (ie 3 days after transfer of P cells). Between 4 and 10 mice per group is adequate for studies proposed here.

(iii) Cause of Relapsing HSE

129-Rag mice infected with HSVDlux and treated with IVIG 24 h PI can be imaged for replicating HSV on day 8 and 12 PI. Mice are treated with (a), IVIG on day 14 PI or (b) ACV daily until day 24 PI or (c) PBS and monitored for survival and by BLI for the presence of replicating HSV on day 16, 20, 24 and 28 PI. Mortality due to relapsing HSE usually begins ˜day 14 PI in these mice. The expectation was that all control mice in group (c) can die before day 28 PI. If immune mechanisms are the dominant cause of death high mortality occurs in group (b). High survival occurs in group (a), at least up to day 28 PI since our data indicate that IVIG can suppress both inflammation and virus replication; thereafter, relapsing HSE is anticipated. This validates the proposed two component model for IVIG protection in 129-mice.

### (iv) In Vivo Localization of Adoptively Transferred P-Cells.

HSV infected 129-Rag mice adoptively transferred with untreated P-cells at 24 h PI and immediately infused with IVIG or infected mice transferred with in vitro IVIG treated P-cells at 24 h PI can be sacrificed on days 2, 4 and 6 PI (4 mice/group/time point=24 mice). Presence of P-cells in blood, dLN, spleen and BS can be assessed by flow cytometry analysis of cells stained for a P-cell specific surface marker while specific localization can be confirmed in paraffin embedded sections stained with P-cell specific mAbs. Optionally, P-cells can be CFSE labeled and proliferation of these cells and accumulation within specific organs can be analyzed by flow cytometry. CFSE can detect cells that have divided 7-8 times. Cytokine secretion by P-cells isolated from lymphoid organs and the CNS can be assessed by intracellular flow cytometry or by ELISPOT assays.

### Purification of B and T Cells for Adoptive Transfer.

Magnetic cell sorting (MACS) with MicroBeads (Miltenyi) can be used to enrich cell populations by positively selecting the “unwanted” cells, thereby negatively selecting and enriching the desired cells as an ‘untouched’ population. Cells are either selected by beads conjugated to a specific antibody recognizing a molecule displayed on their surface, or stained with a fluorescent dye conjugated antibody first and then selected with magnetic beads conjugated with an antibody specifically recognizing the fluorescent dye. Cells are labeled according to the manufacturers protocol (Miltenyi Biotec MACS MicroBeads). In brief, after labeling the cells, the cell suspension is loaded on a column, which is placed in the magnetic field of an AutoMACS separator that is available in the Division of Immunology (PI has an adjunct appointment in Immunology). The magnetically labeled cells are retained in the column while the unlabeled cells are eluted in the void volume—these are the untouched negatively selected cells. Spleen cell suspensions prepared from normal mice can be used for preparing purified donor B and T cell populations for adoptive transfer. Adoptive transfer can be done by tail vein injection of from 5×10⁵-1×10⁷ purified cells (68).

### Determining the Anti-Inflammatory Mechanism of IVIG in HSE

The IVIG ‘sensor’ cell populations can be defined, the requirement for activating and inhibitory Fcg receptor (FcgR) expression in hematopoietic and BS tissues can be determined and involvement of chemokine and cytokine signaling in secondary lymphoid tissues and BS can be investigated. Additionally, studies can be initiated to understand the mechanism by which IVIG suppresses virus replication in neurons.

Anti-inflammatory mechanisms of IVIG have been studied primarily in autoimmune disease models (91). However, much less is known about viral disease models, including models incorporating immunosuppression, a clinical situation in which IVIG is routinely administered (13, 123). Contrasting IVIG protection in WT and immunodeficient Rag/E mice revealed unexpected genetic differences in IVIG mediated protection against fatal HSV induced inflammatory disease (HSE) in different mouse strain backgrounds. A robust model was developed for deciphering IVIG mechanisms of action in protection against HSE (FIG. 15). This model can also provide important insights into differences in IVIG action in immunodeficient animals that are relevant to clinical use of IVIG in severely immunosuppressed individuals including neonates. Several important findings from studies of HSE in 129 mice are: (i), macrophages are important effector cells in HSE, being protective in B6 but pathogenic in 129 mice (manuscript submitted) (ii), activated macrophages comprise >60% of total cells in the BS and ˜75% of cells infiltrating the brains of HSV infected WT 129 mice at day 8 PI (iii), IVIG exerts marked suppression of inflammation such that activated macrophages comprise <2% of cells infiltrating the brain and <5% of total cells in BS (section C) (iv), macrophages isolated from HSV infected 129 mice at (day 6-8 PI) exhibit massive spontaneous degranulation attesting to a highly activated aggressive phenotype but IVIG treatment results in less activated phenotype (FIG. 8), consistent with macrophages being pathogenic effector cells (v), presumptive pDCs (CD11c, B220+ PDCA1+) accumulate in large numbers in the brainstem of IVIG treated infected WT and 129-Rag mice as early as day 8 PI and are retained for extended periods of time and (vi), genes involved in IFN-g signaling (Mig, IP-10, STAT1 and IRF1) are potently upregulated in the neural tissues as early as day 4 PI.

The two-cell model of Ravetch (91) explains anti-inflammatory mechanisms of IVIG but involvement of a third cell type, the P-cell is indicated for protection of 129-Rag but not B6-Rag/E or surprisingly 129-E mice. IVIG, specifically minor fraction of sialylated IgG, activates a sensor cell that in turn increases the threshold for activation of effector macrophages possibly by upregulating expression of the inhibitory FcgRIIb. Since sialylated IgG have reduced affinity for FcRs, activation involves some unknown IgG receptor on sensor cells. The sensor cell is an APC that acquired antigen independently of being infected and thus, it can not express the HSV FcgR that binds human but not rodent IgGs (85). Macrophages and DCs are likely sensor cell candidates and the fact that DCs accumulate in BS of IVIG treated mice makes them especially attractive. The massive activation of inflammatory macrophages in BS evidenced by up-regulation of MHC II expression indicates a role for IFN-g signaling, which encourages speculation that IVIG mediated protection against fatal HSE involves modulation of this pathway. These considerations apply largely to 129 mice since HSV infected B6-Rag mice do not have overt inflammatory responses, IVIG mediated protection in B6-RAG mice involves only suppression of HSV replication.

### Identity of IVIG Sensor and Effector Cell Populations in 129 Mice.

The sensor cell is defined as a cell type that can be primed by in vitro IVIG treatment to exert potent anti-inflammatory effects and block virus replication when transferred into infected WT and 129-Rag mice (at 24 h PI). Although sustained protection is expected in WT mice, only transient protection is expected in 129-Rag mice while sustained protection of 129-E is anticipated. The capacity of in vitro primed 129-Rag spleen cells to protect WT 129 HSV infected mice adoptively transferred at 24 h PI (6 mice/group) can be assessed. Flow sorted splenic F480⁺ macrophages and CD11c⁺ DCs primed in vitro with IVIG can be similarly evaluated; the sensor cell can be identified by its capacity to confer sustained protection to 129 mice (6 mice/group). WT spleen macrophages and DCs primed in vitro with IVIG were tested for protection. DCs and macrophages in WT and 129-Rag mice are functionally equivalent with regards to IVIG protection.

The capacity of sensor cells, treated or not with IVIG, to down regulate spontaneous or HSV antigen induced degranulation and inflammatory cytokine secretion (e.g. TNF-alpha) by macrophages isolated from spleens of HSV infected WT mice at day 6-8 PI can be evaluated. A transwell system can be used to determine whether modulation of effector macrophage phenotype is contact dependent or cytokine mediated. Soluble factors can be identified using RT-PCR arrays probed with total RNA prepared from mixed cultured of IVIG treated or not treated S-cells incubated with aggressive 129 macrophages obtained from spleens of infected mice. PCR results can be further confirmed by either ELISA or flow cytometry and depleted from the system with specific antibodies. The phenotype of macrophages can be examined in the absence of this soluble factor.

### Requirement for FcgR Signaling in IVIG Mediated Protection in the HSE Model.

The inhibitory FcgRIIb has a prominent role in the anti-inflammatory effects of IVIG in autoimmune disease models (89, 103). However, signaling via the activating FcgRIII was recently reported to suppress IFN-g signaling in macrophages thereby contributing to IVIG anti-inflammatory effects (96). The requirement for activating (FcgRI and FcgRIII) and inhibitory (FcgRIIb) FcgRs can be assessed by monitoring (i), expression of FcgRI, FcgRIIb and FcgRIII in dLN and spleen of non-treated and IVIG treated 129, 129-Rag/E and B6-Rag/E mice using real time PCR.

When expression profiles are significantly different, APCs (DC, macrophages and B cells) can be flow sorted and FcgR expression can be compared on these populations (ii), in vitro IVIG effects on FcgR expression on S-cells and P-cells; additionally, FcgR expression on effector macrophages isolated from lymphoid organs and BS of infected untreated or IVIG treated 129 mice on day 8 PI can be compared and (iii), assessing protection of 129 mice (10 mice/group) by adoptively transferred P-cells or in vitro IVIG primed S-cells obtained from FcgRIIb^{-/-}, FcgRIII^{-/-} and FcRn chain-deficient (lack activating FcgRs, PIR-A and NK cell cytotoxicity receptors) mice on the B6 background (Taconic Laboratory). P-cells are transferred into infected 129-Rag mice immediately after IVIG treatment at 24 h PI, while primed S-cells are transferred into WT 129 mice at 24 h PI (10 mice/group). A caveat is that IVIG protection differs fundamentally in B6 and 129 mice raising the possibility the P-cells/S-cells in 129 and B6 mice might differ functionally.

Protection of B6-Rag/E mice involves IVIG mediated suppression of HSV replication, whereas protection of 129-Rag/E mice additionally requires the anti-inflammatory activity of IVIG. If B6 derived in vitro IVIG primed S-cells can mediate protection of WT 129 mice this concern can be abrogated. Survival of HSV infected 129-E/WT mice adoptively transferred with P-cells/S-cells transfected with siRNAs targeting FcgRIIb and FcgRIII can be monitored (10 mice/group). Controls can be P-cells/sensor cells transfected with an irrelevant siRNA (10 mice/group). Efficiency of knockdown can be determined by flow analysis of cells stained for FcgRs. Mirus-TKO transfection reagent was used, which returns transfection efficiencies of >90% with diverse cell lines (eg. HL-60, HeLa, CV-1, RAW264.7, HT4) and primary macrophages.

### A Role for FcgR Signaling in Neurons in IVIG Mediated Suppression of HSV Replication.

Functional FcgRs have been described in normal human CNS and recent studies in mouse and rabbit models show that microglial and neuronal IgG immunoreactivity is due to IgG uptake by FcgRs (98, 135). Microglia having a heamatopoietic origin express FcgRs. FcgR mediated uptake of IgG by motor neurons (38) and certain sensory DRG neurons express the high affinity activating FcgRI. Binding of IgG-antigen complexes increased intracellular Ca2+ ion concentration in DRG neurons and triggered release of substance P via interaction with FcgRI (3). Thus, in IVIG treated mice binding of HSV antigen-antibody complexes by FcgRs on neurons triggers signaling events resulting in suppression of HSV replication. Alternatively, HSV specific IgG binds specific HSV glycoproteins trigger signaling that results in suppression of replication. The apparent requirement for IVIG preparations that recognize HSV antigens makes sense then considering that in several autoimmune models IVIG anti-inflammatory effects requires only the Fc IgG domain (91). A similar situation exists for West Nile virus, since only IVIG preparations from countries where the virus is endemic are protective in normal but not immunodeficient mice (6, 27).

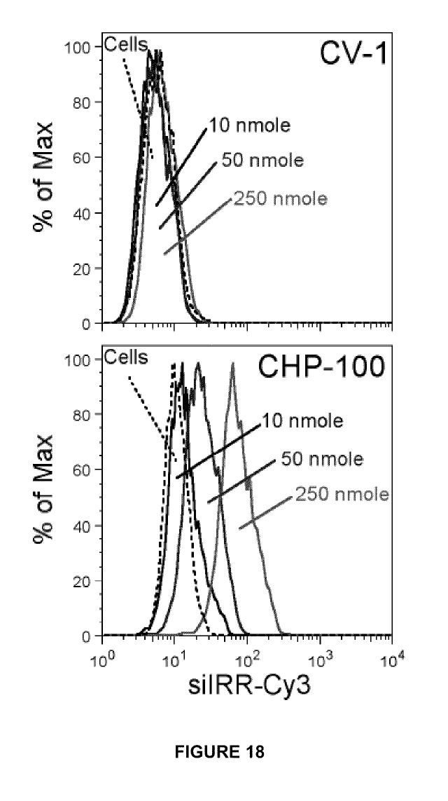

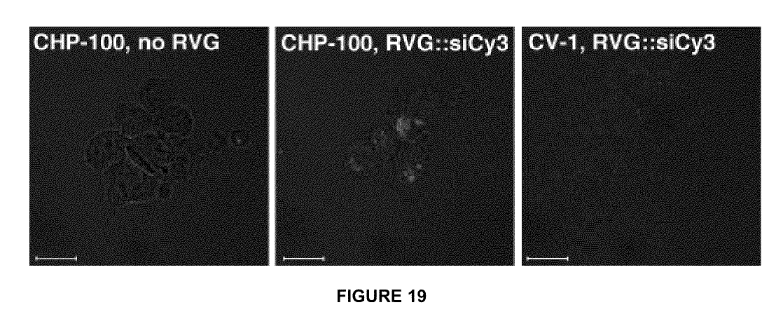

To determine a role for FcgR expression on CNS microglia, neurons and endothelial cells in IVIG mediated protection in 129/B6-Rag mice siRNAs can be used to down regulate expression of FcgR1, FcgRIIb and FcgRIII. A short peptide derived from Rabies virus glycoprotein (RVG) was recently shown to facilitate transvascular delivery of small interfering RNA (siRNA) specifically to the brain. This peptide specifically binds the acetylcholine receptor expressed by neural cells. To enable siRNA binding, a chimeric peptide comprised of nonamer arginine residues added at the C-terminus of the RVG peptide was constructed (RVG-9R). siRNA-RVG-9R complexes specifically targeted neuronal cells in vitro and most importantly were delivered specifically to brain and not liver or lung after IV injection. FIG. 18 shows specific uptake of Cy3-siRNA-RVG-9R by human neuronal CHP cells but not non-neuronal CV1 monkey cells and FIG. 19 shows Cy3-siRNA-RVG-9R to the cytoplamic compartment after uptake. Cy3-siRNA (50 μg) complexed with RVG-9R was injected iv into B6 mice that were sacrificed ˜18 h later.

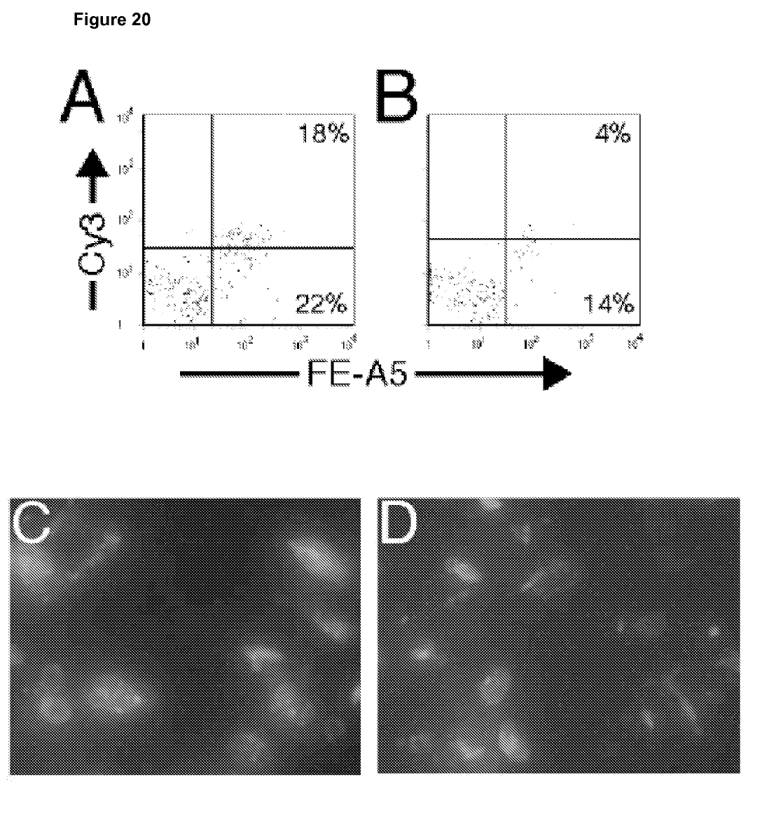

Whole brain single cell suspensions were stained with the A5 mAb that specifically recognizes the neuronal subset in which HSV preferentially establishes latent infection (75). FIG. 20A shows specific delivery of Cy3-siRNA to A5 neurons; only background auto fluorescence is seen with RVG-9R alone (FIG. 20B). Specific delivery of Cy3-siRNA to neurons is shown in cryosections of Tg stained with DAPI to visualize nuclei; large complexes of Cy3-siRNA are seen in the cytoplasm (FIG. 20C) whereas no signal is seen with just the RVG-9R peptide (FIG. 20D). These results confirm that the RVG-9R peptide mediates specific delivery of siRNAs to neurons in the Tg and CNS including neurons in which latency is preferentially established. This system can be exploited to specifically down-regulate FcgR expression in the brain because the RVG peptide is able traverse the intact BBB whereas the rabies virus matrix derived peptide, MAT-9R does not mediate neuronal uptake or penetration of the BBB. B6-Rag mice injected IV with siFcgR-RVG-9R, siFcgR-MAT-9R or siIRR-RVG-9R control complexes can be infected with HSV-GFP 48 h later and then given a second dose of siFcgR-RVG-9R or control complexes. IVIG treatment is given 24 h PI and mice are monitored for survival (10 mice/group).

### IgG Binding to Neurons.

Monomeric IgG can be labeled with Alexa Fluor 594 (590/61, adsorption/emission; red,) using the Xenon human IgG labeling kit (Cat. # Z25407, Molecular Probes, Invitrogen). Alexa fluors are extremely bright photostable fluorophores ideally suited for imaging applications. Alexa-labeled IgG can be given to mock or HSV-GFP infected B6-Rag mice at 24 h PI. Tg cryosections (6-8 μM) prepared from mice sacrificed at 48 and 72 h PI can be examined by confocal microscopy for IgG binding to neurons that if infected can be identified by GFP expression. B6-Rag mice are studied because IVIG protection in these mice involves only suppression of virus replication. Monomeric IgG can be labeled in the hinge region since this significantly enhances the fluorescent signal. The binding of F′(ab)₂ fragments can be compared to determine whether antigen recognition is involved. Binding of F′(ab)₂ fragments on neurons can be detected using an Alexa-conjugated 2 antibody specific for human F′(ab)₂. Tg neurons are studied because they are easily accessible and viable in culture for several days. Dissociated Tg cultures established from HSV-GFP infected mice can be incubated with or without IVIG at different concentrations (0.1, 0.5 and 1.0 mg/ml) and HSV replication can be monitored indirectly by fluorescence microscopy. Real time PCR for HSV genome load can be done on cultures harvested after 3-5 days incubation to assay for replication of HSV DNA.

### A Role for IFN-g Signaling in IVIG Mediated Protection.

IVIG treatment of HSV infected IFN-g null mutant 129-mice (Gko) can reveal if IFN-g signaling is required for induction of protective responses (6 mice/group). IVIG protection of Gko mice indicates that IFN-g is not required. The IFN-g null mutation in the pure 129 background was rederived, averting potential problems from differences in strain background. (11). Observing down regulation of IFN-g and/or its receptor in IVIG treated mice (Aim 1) implies that suppression of IFN-g signaling is required for IVIG protection (96). If indicated, in vitro effects of IVIG on IFN-g and IFN-gR expression in P-cells/S-cells can be determined by RT-PCR and confirmed by flow cytometry using anti-IFN-g R2 mAb. The requirement for IFN-gR expression on hematopoietic cells can be discriminated versus non-hematopoietic cells using reciprocal bone marrow (bm) chimeras between 129 and 129-IFN-gR^{-/-} (Rgko) mice (10). Lethally irradiated 129 mice reconstituted with bm from Rgko mice express the IFN-gR only on non hematopoietic cells, while the converse is true for lethally irradiated Rgko mice reconstituted with WT bm. Studying IVIG mediated protection against HSE in these chimeras can reveal whether IFN-g signaling is required in heamatopoietic or non-heamatopoietic cells. IFN-g signaling is required for IVIG anti-inflammatory effects or suppression of replication by monitoring these two parameters in brainstems of IVIG treated infected mice at day 6 PI. These results can also be confirmed by adoptively transferring P-cells purified from IFN-gKO mice into HSV infected 129 RAG mice treated with IVIG and their effect on virus replication determined. Alternatively, S-cells derived from IFN-Rgko donor mice can be transferred into HSV infected 129 RAG mice and their influence on inflammation within the BS compared to control HSV infected 129 RAG mice by flow cytometry at day 6-8 PI. These two experiments can reveal the role of IFN—if any, on either virus replication or modulation of CNS inflammation. On going studies indicate a role for IFN-g in some aspect of IVIG protection in the HSE model.

### Gene Expression Profiling in 129- and B6-Rag Mice and P-Cell Transferred 129-Rag Mice.

The present invention is to determine changes in expression of cytokines, chemokines and their associated receptors in dLN, BS and individual Tg neurons of 129-Rag and B6-Rag mice. Tg neurons are chosen because they are large, easily accessible and are the recognized site of latent HSV infection.

129/B6-Rag mice inoculated with HSV-GFP are treated or not treated with IVIG at 24 h PI; mock infected mice treated or not treated with IVIG can serve as controls (24 mice/group). Mice can be sacrificed at 24 and 72 h after IVIG infusion (12 mice per time point) and the following assays can be done: (A), microarray analysis for expression of cytokines/chemokines and their receptors—pooled dLN cells and BS from 4 mice (B), microarray analysis on infected pooled neurons (identified by GFP expression)—˜60 pooled neurons captured by laser capture microdissection (LCM) from 4 Tg (C), real time PCR and flow cytometric staining to measure FcgR expression—total RNA prepared from 6 pooled Tg and 3 pooled BS (D), Taqman PCR for HSV DNA load in 6 individual Tg and 3 individual BS. A total of 192 mice can be required. Possible differences in expression of IFN response genes, cytokine and chemokine genes in B6/129-Rag mice in response to IVIG treatment can be revealed.

129-Rag mice inoculated with HSV-GFP and transferred ˜6 h PI with P-cells or not transferred can be treated or not treated with IVIG at 24 h PI; mock infected mice treated or not with IVIG can serve as controls (laser microdissection). Mice sacrificed at 24 and 72 h after IVIG treatment (12 mice per time point) can be analyzed as in (a). P-cell induced changes in expression of innate response genes that result in robust IVIG protection.

IVIG induced signaling via the activating FcgRIII has been reported to suppress IFN-g signaling in macrophages thereby contributing to IVIG's anti-inflammatory effects in an IFN-g enhanced model of ITP (95). As shown in FIG. 13, IVIG protection is severely impaired in HSV infected IFN-g^{-/} (Gko) mice that were rederived on the 129-strain background (11). Examination of IVIG treated Gko mice revealed that while HSV CNS replication was suppressed normally by day 8-10 pi, exaggerated CNS inflammation persisted. Hyperinflammatory responses were maintained in surviving mice sacrificed at day 18 pi. These results indicate that IFN-g the prototypical proinflammatory cytokine, is preferred for IVIG's anti-inflammatory activity in a HSE model.

### Laser Capture Microdissection

HSV infected neurons expressing GFP can be isolated from frozen fixed Tg sections by laser microdissection and pressure catapulting (LMPC) using the PALM MicroBeam System, (Carl Zeiss). LMPC can be used for capture of Tg neurons. RNA can be extracted from pooled captured neurons (30-60) from each group of Tg using the RNAqueous kit (Ambion, Inc., Austin Tex.) specifically designed for isolating high quality RNA from LCM samples or small numbers of cells. Total RNA can be amplified using the novel highly efficient Ovation Aminoallyl RS system (NuGEN Technologies Inc., San Carlos Calif.) to produce fluorescently labeled cDNA for hybridization to microarrays. Advantages of the RS system include production of cDNA rather the less stable cRNA from T-7 amplification systems, relatively simple and rapid to perform and requiring only 5 ng RNA (116). Importantly, recent publications have shown that routine RNA amplification actually improves the quality of microarray results by providing more array-to-array consistency (31, 102) and this is true also for real time PCR using limited amounts of template. The approach here is supported by the rapidly increasing successful use of RNA provided by LCM, needle biopsy or limited numbers of highly purified cells for gene discovery in a variety of experimental settings (37, 47, 48, 105). A protocol for using LCM with in situ hybridization and real-time PCR has been described and was used to study the distribution of latent HSV and VZV genomes in human Tg illustrating the utility of LCM (17, 127). Differential gene expression can be verified by real-time PCR using GAPDH as a reference (57, 68). Based on the observation that genes involved in the interferon pathway are upregulated during acute infection as well as during latency and reactivation (52), genes in this pathway are differentially expressed in 129 and B6 Tg and further that IVIG treatment might influence expression of these genes.

The COH Pathology Core has a Zeiss PALM Laser dissecting microscope that utilizes a UV laser pulsed microbeam for dissection. Snap frozen Tgs stored at −80° C. can be cut in 10-15 mm serial sections on a cryotome at −25° C., transferred to PALM membrane slides (to facilitate the laser pressure catapulting process) and air dried for 10 seconds. After fixation for 5 minutes in 70% ethanol at −20° C. and a 10 second rinse in RNAse free water the sections are counterstained with H&E to monitor tissue integrity. Fluorescence optics can permit identification of GFP expressing neurons that can be excised and catapulted contact free by laser pressure into collecting caps containing 12-40 ml RNAse free water. The isolated neurons can be mixed with “RNAqueous-Micro” lysis buffer developed for RNA extraction from microdissected samples or small numbers of cells (Ambion Inc., Austin, Tex.). The RNA can then be amplified for microarray analysis using the Ovation RS system (NuGEN Technologies, Inc.) or other system recommended by the Functional Genomics Core Laboratory.

The foregoing examples and methods of the invention are illustrative only and are not intended to be limiting of the invention in any way. Those of ordinary skill in the art will recognize that various modifications of the foregoing are within the intended scope of the invention.

All references are incorporated by reference in their entirety as though fully set forth therein.

### REFERENCES

1. Altmann, D. M., and W. A. Blyth. 1985. Protection from herpes simplex virus-induced neuropathology in mice showing delayed hypersensitivity tolerance. J Gen Virol 66:1297-303.
2. Altmann, D. M., and W. A. Blyth. 1985. The effects of cyclosporin A on the induction, expression and regulation of the immune response to herpes simplex virus. Clin Exp Immunol 59:17-22.
3. Andoh, T., and Y. Kuraishi. 2003. Direct action of immunoglobulin G on primary sensory neurons through Fc gamma receptor. FASEB J.:02-1169fje.
4. Baron, J. L., L. Gardiner, S. Nishimura, K. Shinkai, R. Locksley, and D. Ganem. 2002. Activation of a Nonclassical NKT Cell Subset in a Transgenic Mouse Model of Hepatitis B Virus Infection. Immunity 16:583-594.
5. Bell, S., M. Cranage, L. Borysiewicz, and T. Minson. 1990. Induction of immunoglobulin G Fc receptors by recombinant vaccinia viruses expressing glycoproteins E and I of herpes simplex virus type 1. J Virol 64:2181-6.
6. Ben-Nathan, D., S. Lustig, G. Tam, S. Robinzon, S. Segal, and B. Rager-Zisman. 2003. Prophylactic and therapeutic efficacy of human intravenous immunoglobulin in treating West Nile virus infection in mice. J Infect Dis 188:5-12.
7. Bruhns, P., A. Samuelsson, J. W. Pollard, and J. V. Ravetch. 2003. Colony-Stimulating Factor-1-Dependent Macrophages Are Responsible for IVIG Protection in Antibody-induced Autoimmune Disease. Immunity 18:573-581.
8. Budt, M., H. Reinhard, A. Bigl, and H. Hengel. 2004. Herpesviral Fc[gamma] receptors: culprits attenuating antiviral IgG? International Immunopharmacology 4:1135-1148.
9. Cagnin, A., R. Myers, R. N. Gunn, A. D. Lawrence, T. Stevens, G. W. Kreutzberg, T. Jones, and R. B. Banati. 2001. In vivo visualization of activated glia by [11C] (R)-PK11195-PET following herpes encephalitis reveals projected neuronal damage beyond the primary focal lesion. Brain 124, no 10:2014-2027.
10. Cantin, E., B. Tanamachi, and H. Openshaw. 1999. Role for gamma interferon in control of herpes simplex virus type 1 reactivation. J Virol 73:3418-23.
11. Cantin, E., B. Tanamachi, H. Openshaw, J. Mann, and K. Clarke. 1999. Gamma interferon (IFN-gamma) receptor null-mutant mice are more susceptible to herpes simplex virus type 1 infection than IFN-gamma ligand null-mutant mice. J Virol 73:5196-200.
12. Cantin, E. M., D. R. Hinton, J. Chen, and H. Openshaw. 1995. Gamma interferon expression during acute and latent nervous system infection by herpes simplex virus type 1. J. Virol. 69:4898-4905.
13. Casadevall, A., E. Dadachova, and L.-a. Pirofski. 2004. PASSIVE ANTIBODY THERAPY FOR INFECTIOUS DISEASES. Nature Reviews Microbiology 2:695-703.
14. Casadevall, A., and L. A. Pirofski. 2003. Microbial virulence results from the interaction between host and microorganism. Trends Microbiol 11:157-8; author reply 158-9.
15. Casadevall, A., and L. A. Pirofski. 2003. The damage-response framework of microbial pathogenesis. Nat Rev Microbiol 1:17-24.
16. Chaudhuri, A., and P. G. E. Kennedy. 2002. Diagnosis and treatment of viral encephalitis. Postgrad Med J 78:575-583.
17. Chen, X. P., M. Mata, and D. J. Fink. 2005. Use of laser capture microdissection together with in situ hybridization and real-time PCR to study distribution of latent herpes simplex virus genomes in mouse trigeminal ganglia. Methods Mol Biol 293:285-93.
18. Cho, C. T., K. K. Feng, and N. Brahmacupta. 1976. Synergistic antiviral effects of adenine arabinoside and humoral antibodies in experimental encephalitis due to Herpesvirus hominis. J Infect Dis 133:157-67.
19. Clynes, R. 2007. IVIG Therapy: Interfering with Interferon-[gamma]. Immunity 26:4-6.
20. Clynes, R. 2007. Protective mechanisms of IVIG. Current Opinion in Immunology 19:646-651.
21. Coen, D. M. 1991. The implications of resistance to antiviral agents for herpesvirus drug targets and drug therapy. Antiviral Res 15:287-300.
22. Dalai, S. K., L. Pesnicak, G. F. Miller, and S. E. Straus. 2002. Prophylactic and therapeutic effects of human immunoglobulin on the pathobiology of HSV-1 infection, latency, and reactivation in mice. Journal of Neurovirology 8:35-44.
23. Debre, M., M. C. Bonnet, W. H. Fridman, E. Carosella, N. Philippe, P. Reinert, E. Vilmer, C. Kaplan, J. L. Teillaud, and C. Griscelli. 1993. Infusion of Fc gamma fragments for treatment of children with acute immune thrombocytopenic purpura. Lancet 342:945-9.
24. Dix, R. D., L. Pereira, and J. R. Baringer. 1981. Use of monoclonal antibody directed against herpes simplex virus glycoproteins to protect mice against acute virus-induced neurological disease. Infect Immun 34:192-9.
25. Dubin, G., I. Frank, and H. M. Friedman. 1990. Herpes simplex virus type 1 encodes two Fc receptors which have different binding characteristics for monomeric immunoglobulin G (IgG) and IgG complexes. J Virol 64:2725-31.
26. Dudl, E., A. Toyama, C. Arber, A. BitMansour, J. Brown, B. Chung, M. Price, J. M. Y. Brown, and K. I. Weinberg. 2004. Common lymphoid progenitors require common gamma ([gamma]c) and C-kit signals for survival in vivo. Biology of Blood and Marrow Transplantation 10:82-83.
27. Engle, M. J., and M. S. Diamond. 2003. Antibody prophylaxis and therapy against West Nile virus infection in wild-type and immunodeficient mice. J Virol 77:12941-9.
28. Erlich, K. S., R. D. Dix, and J. Mills. 1987. Prevention and treatment of experimental herpes simplex virus encephalitis with human immune serum globulin. Antimicrob Agents Chemother 31:1006-9.
29. Erlich, K. S., and J. Mills. 1986. Passive immunotherapy for encephalitis caused by herpes simplex virus. Rev Infect Dis 8 Suppl 4:S439-45.
30. Esiri, M. M., and P. G. E. Kennedy. 1997. Viral Diseases, p. 3-63. In D. L. Graham and P. L. Lantos (ed.), Greenfield's Neuropathology, 6th Edition, vol. 2. Arnold.
31. Feldman, A. L., N. G. Costouros, E. Wang, M. Qian, F. M. Marincola, H. R. Alexander, and S. K. Libutti. 2002. Advantages of mRNA amplification for microarray analysis. Biotechniques 33:906-12, 914.
32. Frank, I., and H. M. Friedman. 1989. A novel function of the herpes simplex virus type 1 Fc receptor: participation in bipolar bridging of antiviral immunoglobulin G. J Virol 63:4479-88.
33. Fry, T. J., and C. L. Mackall. 2001. Interleukin-7: master regulator of peripheral T-cell homeostasis? Trends in Immunology 22:564-71.
34. Galli, S. J., S. Nakae, and M. Tsai. 2005. Mast cells in the development of adaptive immune responses. 6:135-142.
35. Ghiasi, H., R. Kaiwar, A. B. Nesburn, S. Slanina, and S. L. Wechsler. 1994. Expression of seven herpes simplex virus type 1 glycoproteins (gB, gC, gD, gE, gG, gH, and gI): comparative protection against lethal challenge in mice. Journal Of Virology 68:2118-26.
36. Goldsmith, S. M., and R. Whitley. 1991. Herpes simplex encephalitis. B. C. Decker, Philadelphia.
37. Gustincich, S., M. Contini, M. Gariboldi, M. Puopolo, K. Kadota, H. Bono, J. LeMieux, P. Walsh, P. Carninci, Y. Hayashizaki, Y. Okazaki, and E. Raviola. 2004. Gene discovery in genetically labeled single dopaminergic neurons of the retina. Proc Natl Acad Sci USA 101:5069-74.
38. Habib A. Mohamed, et al. 2002. Immunoglobulin Fc? receptor promotes immunoglobulin uptake, immunoglobulin-mediated calcium increase, and neurotransmitter release in motor neurons. Journal of Neuroscience Research 69:110-116.
39. Hayashida, I., S. Nagafuchi, Y. Hayashi, Y. Kino, R. Mori, H. Oda, N. Ohtomo, and A. Tashiro. 1982. Mechanism of antibody-mediated protection against herpes simplex virus infection in athymic nude mice: requirement of Fc portion of antibody. Microbiol Immunol 26:497-509.
40. Hirokazu, H., M. Tadashi, K. Makoto, Y. Tasuku, U. Koji, and M. Akira. 2004. Roles of mast cells in the pathogenesis of viral myocarditis; reconstitution of mast cells to their deficient mice. Journal of Cardiac Failure 10:S162.
41. Ito, Y., H. Kimura, Y. Yabuta, Y. Ando, T. Murakami, M. Shiomi, and T. Morishima. 2000. Exacerbation of Herpes Simplex Encephalitis after Successful Treatment with Acyclovir. Clinical infectious diseases: an official publication of the Infectious Diseases Society of America 30:185 (3 pages).
42. Jefferis, R. 2006. A sugar switch for anti-inflammatory antibodies. Nat Biotechnol 24:1230-1.
43. Jefferis, R., and J. Lund. 2002. Interaction sites on human IgG-Fc for FcgammaR: current models. Immunol Lett 82:57-65.
44. John Looney, R., and J. Huggins. 2006. Use of intravenous immunoglobulin G (IVIG). Best Practice & Research Clinical Haematology 19:3-25.
45. Jones, C. 1998. Alphaherpesvirus latency: its role in disease and survival of the virus in nature. Adv Virus Res 51:81-133.
46. Joos, Ziyeh, Rauer, Keller, Huzly, and Licking. 2003. Postinfectious Autoimmune-Mediated Encephalitis Eight Months after Herpes simplex Encephalitis. European Neurology 50, no 1:54-56.
47. Kamme, F., and M. G. Erlander. 2003. Global gene expression analysis of single cells. Curr Opin Drug Discov Devel 6:231-6.
48. Kamme, F., R. Salunga, J. Yu, D. T. Tran, J. Zhu, L. Luo, A. Bittner, H. Q. Guo, N. Miller, J. Wan, and M. Erlander. 2003. Single-cell microarray analysis in hippocampus CA1: demonstration and validation of cellular heterogeneity. J Neurosci 23:3607-15.
49. Kaneko, Y., F. Nimmerjahn, M. P. Madaio, and J. V. Ravetch. 2006. Pathology and protection in nephrotoxic nephritis is determined by selective engagement of specific Fc receptors. Journal of Experimental Medicine 203:789-797.
50. Kaneko, Y., F. Nimmerjahn, and J. V. Ravetch. 2006. Anti-inflammatory Activity of Immunoglobulin G Resulting from Fc Sialylation. Science 313:670-673.
51. Kang, J., M. Coles, and D. H. Raulet. 1999. Defective development of gamma/delta T cells in interleukin 7 receptor-deficient mice is due to impaired expression of T cell receptor gamma genes. The Journal of Experimental Medicine 190:973-82.
52. Kent, J. R., and N. W. Fraser. 2005. The cellular response to herpes simplex virus type 1 (HSV-1) during latency and reactivation. J Neurovirol 11:376-83.
53. Kimbrell, D. A., and B. Beutler. 2001. The evolution and genetics of innate immunity. Nat Rev Genet 2:256-67.
54. Kodukula, P., T. Liu, N. V. Rooijen, M. J. Jager, and R. L. Hendricks. 1999. Macrophage control of herpes simplex virus type 1 replication in the peripheral nervous system. J Immunol 162:2895-905.
55. Kohl, S., N. C. Strynadka, R. S. Hodges, and L. Pereira. 1990. Analysis of the role of antibody-dependent cellular cytotoxic antibody activity in murine neonatal herpes simplex virus infection with antibodies to synthetic peptides of glycoprotein D and monoclonal antibodies to glycoprotein B. J Clin Invest 86:273-8.
56. Koskiniemi, M., H. Piiparinen, L. Mannonen, T. Rantalaiho, and A. Vaheri. 1996. Herpes encephalitis is a disease of middle aged and elderly people: polymerase chain reaction for detection of herpes simplex virus in the CSF of 516 patients with encephalitis. The Study Group. J Neurol Neurosurg Psychiatry 60:174-8.
57. Kramer, M. F., W. J. Cook, F. P. Roth, J. Zhu, H. Holman, D. M. Knipe, and D. M. Coen. 2003. Latent Herpes Simplex Virus Infection of Sensory Neurons Alters Neuronal Gene Expression. J. Virol. 77:9533-9541.
58. Lang, A., and J. Nikolich-Zugich. 2005. Development and Migration of Protective CD8+ T Cells into the Nervous System following Ocular Herpes Simplex Virus-1 Infection. J Immunol 174:2919-2925.
59. Lantz, C. S., J. Boesiger, C. H. Song, N. Mach, T. Kobayashi, R. C. Mulligan, Y. Nawa, G. Dranoff, and S. J. Galli. 1998. Role for interleukin-3 in mast-cell and basophil development and in immunity to parasites. Nature 392:90-3.
60. LeBlanc, R. A., L. Pesnicak, M. Godleski, and S. E. Straus. 1999. Treatment of HSV-1 infection with immunoglobulin or acyclovir: comparison of their effects on viral spread, latency, and reactivation [In Process Citation]. Virology 262:230-6.
61. Lellouch-Tubiana, A., M. Fohlen, O. Robain, and F. Rozenberg. 2000. Immunocytochemical characterization of long-term persistent immune activation in human brain after herpes simplex encephalitis. Neuropathology and Applied Neurobiology 26:285-94.
62. Lemm, G. 2002. Composition and properties of IVIg preparations that affect tolerability and therapeutic efficacy. Neurology 59:S28-32.
63. Levine, B., J. M. Hardwick, B. D. Trapp, T. O. Crawford, R. C. Bollinger, and D. E. Griffin. 1991. Antibody-mediated clearance of alphavirus infection from neurons. Science 254:856-60.
64. Li, H., N. Sethuraman, T. A. Stadheim, D. Zha, B. Prinz, N. Ballew, P. Bobrowicz, B.-K. Choi, W. J. Cook, M. Cukan, N. R. Houston-Cummings, R. Davidson, B. Gong, S. R. Hamilton, J. P. Hoopes, Y. Jiang, N. Kim, R. Mansfield, J. H. Nett, S. Rios, R. Strawbridge, S. Wildt, and T. U. Gerngross. 2006. Optimization of humanized IgGs in glycoengineered Pichia pastoris. Nat Biotech 24:210-215.
65. Liu, T., Q. Tang, and R. L. Hendricks. 1996. Inflammatory infiltration of the trigeminal ganglion after herpes simplex virus type 1 corneal infection. J Virol 70:264-71.
66. Luker, G. D., J. P. Bardill, J. L. Prior, C. M. Pica, D. Piwnica-Worms, and D. A. Leib. 2002. Noninvasive Bioluminescence Imaging of Herpes Simplex Virus Type 1 Infection and Therapy in Living Mice. J. Virol. 76:12149-12161.
67. Lundberg, P., H. Openshaw, M. Wang, H. J. Yang, and E. Cantin. 2007. Effects of CXCR3 Signaling on Development of Fatal Encephalitis and Corneal and Periocular Skin Disease in HSV-Infected Mice Are Mouse-Strain Dependent. Invest Opthalmol Vis Sci 48:4162-70.
68. Lundberg, P., P. Welander, H. Openshaw, C. Nalbandian, C. Edwards, L. Moldawer, and E. Cantin. 2003. A locus on mouse chromosome 6 that determines resistance to herpes simplex virus also influences reactivation, while an unlinked locus augments resistance of female mice. J Virol 77:11661-73.
69. Lundberg, P., P. V. Welander, C. K. Edwards, III, N. van Rooijen, and E. Cantin. 2007. Tumor Necrosis Factor (TNF) Protects Resistant C57BL/6 Mice against Herpes Simplex Virus-induced Encephalitis Independently of Signaling via TNF Receptor 1 or 2. J. Virol. 81:1451-1460.
70. Margolis, T. P., F. Sedarati, A. T. Dobson, L. T. Feldman, and J. G. Stevens. 1992. Pathways of viral gene expression during acute neuronal infection with HSV-1. Virology 189:150-60.
71. Masci, S., C. De Simone, G. Famularo, M. Gravante, M. Ciancarelli, M. Andreassi, P. Amerio, and G. Santini. 1995. Intravenous immunoglobulins suppress the recurrences of genital herpes simplex virus: a clinical and immunological study. Immunopharmacol Immunotoxicol 17:33-47.
72. McKendall, R. R. 1985. IgG-mediated viral clearance in experimental infection with herpes simplex virus type 1: role for neutralization and Fc-dependent functions but not C′ cytolysis and C5 chemotaxis. J Infect Dis 151:464-70.
73. Mekori, Y. A., and D. D. Metcalfe. 2000. Mast cells in innate immunity. Immunological Reviews 173:131-40.
74. Mester, J. C., J. C. Glorioso, and B. T. Rouse. 1991. Protection against zosteriform spread of herpes simplex virus by monoclonal antibodies. J Infect Dis 163:263-9.
75. Metcalfe, D. D., D. Baram, and Y. A. Mekori. 1997. Mast cells. Physiol Rev 77:1033-79.
76. Meyding-Lamade, U., W. Lamade, R. Kehm, C. Oberlinner, A. Fath, B. Wildemann, J. Haas, and W. Hacke. 1999. Herpes simplex virus encephalitis: chronic progressive cerebral MRI changes despite good clinical recovery and low viral load—an experimental mouse study. 6:531-8.
77. Meyding-Lamade, U. K., W. R. Lamade, B. T. Wildemann, K. Sartor, and W. Hacke. 1999. Herpes simplex virus encephalitis: chronic progressive cerebral magnetic resonance imaging abnormalities in patients despite good clinical recovery. Clinical Infectious Diseases 28:148-9.
78. Meyding-Lamade, U. K., C. Oberlinner, P. R. Rau, S. Seyfer, S. Heiland, J. Sellner, B. T. Wildemann, and W. R. Lamade. 2003. Experimental herpes simplex virus encephalitis: a combination therapy of acyclovir and glucocorticoids reduces long-term magnetic resonance imaging abnormalities. J Neurovirol 9:118-25.
79. Mikloska, Z., P. P. Sanna, and A. L. Cunningham. 1999. Neutralizing antibodies inhibit axonal spread of herpes simplex virus type 1 to epidermal cells in vitro. J Virol 73:5934-44.
80. Miller, C. L., V. I. Rebel, C. D. Helgason, P. M. Lansdorp, and C. J. Eaves. 1997. Impaired steel factor responsiveness differentially affects the detection and long-term maintenance of fetal liver hematopoietic stem cells in vivo. Blood 89:1214-23.
81. Minagawa, H., and Y. Yanagi. 2000. Latent herpes simplex virus-1 infection in SCID mice transferred with immune CD4+ T cells: a new model for latency. Arch Virol 145:2259-72.
82. Mokhtarian, F., and D. E. Griffin. 1984. The role of mast cells in virus-induced inflammation in the murine central nervous system. Cell Immunol 86:491-500.
83. Mokhtarian, F., S. L. Wesselingh, S. Choi, A. Maeda, D. E. Griffin, R. A. Sobel, and D. Grob. 1996. Production and role of cytokines in the CNS of mice with acute viral encephalomyelitis. J. Neuroimmunology 66:11-22.
84. Nagashunmugam, T., J. Lubinski, L. Wang, L. T. Goldstein, B. S. Weeks, P. Sundaresan, E. H. Kang, G. Dubin, and H. M. Friedman. 1998. In vivo immune evasion mediated by the herpes simplex virus type 1 immunoglobulin G Fc receptor. J Virol 72:5351-9.
85. Negi, V.-S., S. Elluru, S. SibÉRil, S. Graff-Dubois, L. U. C. Mouthon, M. D. Kazatchkine, S. Lacroix-Desmazes, J. Bayry, and S. V. Kaveri. 2007. Intravenous Immunoglobulin: An Update on the Clinical Use and Mechanisms of Action. Journal of Clinical Immunology 27:233-245.
86. Nicolaidou, P., N. Iacovidou, S. Youroukos, T. Liacopoulou-Tsitsipi, and C. Kattamis. 1993. Relapse of herpes simplex encephalitis after acyclovir therapy. European journal of pediatrics 152:737.
87. Nimmerjahn, F., and J. V. Ravetch. 2007. Antibodies, Fc receptors and cancer. Curr Opin Immunol 19:239-45.
88. Nimmerjahn, F., and J. V. Ravetch. 2006. Fc[gamma] Receptors: Old Friends and New Family Members. Immunity 24:19-28.
89. Nimmerjahn, F., and J. V. Ravetch. 2008. Fcgamma receptors as regulators of immune responses. Nat Rev Immunol 8:34-47.
90. Nimmerjahn, F., and J. V. Ravetch. 2007. The antiinflammatory activity of IgG: the intravenous IgG paradox. J Exp Med 204:11-5.
91. Nocka, K., J. C. Tan, E. Chiu, T. Y. Chu, P. Ray, P. Traktman, and P. Besmer. 1990. Molecular bases of dominant negative and loss of function mutations at the murine c-kit/white spotting locus: W37, Wv, W41 and W. Embo J 9:1805-13.
92. Oakes, J. E., W. B. Davis, J. A. Taylor, and W. A. Weppner. 1980. Lymphocyte reactivity contributes to protection conferred by specific antibody passively transferred to herpes simplex virus-infected mice. Infect Immun 29:642-9.
93. Oakes, J. E., and R. N. Lausch. 1981. Role of Fc fragments in antibody-mediated recovery from ocular and subcutaneous herpes simplex virus infections. Infect Immun 33:109-14.
94. Park, M. K., D. Amichay, P. Love, E. Wick, F. Liao, A. Grinberg, R. L. Rabin, H. H. Zhang, S. Gebeyehu, T. M. Wright, A. Iwasaki, Y. Weng, J. A. DeMartino, K. L. Elkins, and J. M. Farber. 2002. The CXC Chemokine Murine Monokine Induced by IFN-{gamma} (CXC Chemokine Ligand 9) Is Made by APCs, Targets Lymphocytes Including Activated B Cells, and Supports Antibody Responses to a Bacterial Pathogen In Vivo. J Immunol 169:1433-1443.
95. Park-Min, K.-H., N. V. Serbina, W. Yang, X. Ma, G. Krystal, B. G. Neel, S. L. Nutt, X. Hu, and L. B. Ivashkiv. 2007. Fc[gamma]RIII-Dependent Inhibition of Interferon-[gamma] Responses Mediates Suppressive Effects of Intravenous Immune Globulin. Immunity 26:67-78.
96. Pasquier, B., P. Launay, Y. Kanamaru, I. C. Moura, S. Pfirsch, C. Ruffie, D. Henin, M. Benhamou, M. Pretolani, U. Blank, and R. C. Monteiro. 2005. Identification of Fc[alpha]RI as an Inhibitory Receptor that Controls Inflammation: Dual Role of FcR[gamma] ITAM. Immunity 22:31-42.
97. Peress, N. S., J. Siegelman, H. B. Fleit, M. W. Fanger, and E. Perillo. 1989. Monoclonal antibodies identify three IgG Fc receptors in normal human central nervous system. Clin Immunol Immunopathol 53:268-80.
98. Peschon, J. J., P. J. Morrissey, K. H. Grabstein, F. J. Ramsdell, E. Maraskovsky, B. C. Gliniak, L. S. Park, S. F. Ziegler, D. E. Williams, C. B. Ware, and et al. 1994. Early lymphocyte expansion is severely impaired in interleukin 7 receptor-deficient mice. J Exp Med 180:1955-60.
99. Phares, T. W., R. B. Kean, T. Mikheeva, and D. C. Hooper. 2006. Regional differences in blood-brain barrier permeability changes and inflammation in the apathogenic clearance of virus from the central nervous system. J Immunol 176:7666-75.
100. Planting a, E. G., and J. A. L. Vanneste. 2001. Mild herpes simplex encephalitis worsening despite acyclovir treatment. Journal of neurology Zeitschrift f,r Neurologie. 248:237 (4 pages).
101. Polacek, D. C., A. G. Passerini, C. Shi, N. M. Francesco, E. Manduchi, G. R. Grant, S. Powell, H. Bischof, H. Winkler, C. J. Stoeckert, Jr., and P. F. Davies. 2003. Fidelity and enhanced sensitivity of differential transcription profiles following linear amplification of nanogram amounts of endothelial mRNA. Physiol. Genomics 13:147-156.
102. Ravetch, J. V., and L. L. Lanier. 2000. Immune inhibitory receptors. Science 290:84-9.
103. Rector, J. T., R. N. Lausch, and J. E. Oakes. 1982. Use of monoclonal antibodies for analysis of antibody-dependent immunity to ocular herpes simplex virus type 1 infection. Infect Immun 38:168-74.
104. Reilly, S. C., A. R. Cossins, J. P. Quinn, and L. U. Sneddon. 2004. Discovering genes: the use of microarrays and laser capture microdissection in pain research. Brain Research Reviews 46:225-233.
105. Reith, A. D., R. Rottapel, E. Giddens, C. Brady, L. Forrester, and A. Bernstein. 1990. W mutant mice with mild or severe developmental defects contain distinct point mutations in the kinase domain of the c-kit receptor. Genes Dev 4:390-400.
106. Rivera, J., and A. Casadevall. 2005. Mouse Genetic Background Is a Major Determinant of Isotype-Related Differences for Antibody-Mediated Protective Efficacy against Cryptococcus neoformans. J Immunol 174:8017-8026.
107. Roizman, B., and A. E. Sears. 1987. An inquiry into the mechanisms of herpes simplex virus latency. Annu. Rev. Microbiol. 41:543-577.
108. Roizman, B., and R. J. Whitley. 2001. The nine ages of herpes simplex virus. Herpes 8:23-7.
109. Rouse, B. T. 1996. Virus-induced immunopathology. Adv Virus Res 47:353-76.
110. Sanna, P. P., A. De Logu, R. A. Williamson, Y. L. Hom, S. E. Straus, F. E. Bloom, and D. R. Burton. 1996. Protection of nude mice by passive immunization with a type-common human recombinant monoclonal antibody against HSV. Virology 215:101-6.
111. Sanna, P. P., T. J. Deerinck, and M. H. Ellisman. 1999. Localization of a Passively Transferred Human Recombinant Monoclonal Antibody to Herpes Simplex Virus Glycoprotein D to Infected Nerve Fibers and Sensory Neurons In Vivo. J. Virol. 73:8817-8823.
112. Schneweis, K. E., M. Brado, B. Ebers, A. Friedrich, M. Olbrich, and W. Schuler. 1988. Immunological mechanisms giving rise to latency of herpes simplex virus in the spinal ganglia of the mouse. Med Microbiol Immunol (Berl) 177:1-8.
113. Sciammas, R., P. Kodukula, Q. Tang, R. L. Hendricks, and J. A. Bluestone. 1997. T cell receptor-gamma/delta cells protect mice from herpes simplex virus type 1-induced lethal encephalitis. J Exp Med 185:1969-75.
114. Singh, N. J., and R. H. Schwartz. 2006. The lymphopenic mouse in immunology: from patron to pariah. Immunity 25:851-5.
115. Singh, R., R. J. Maganti, S. V. Jabba, M. Wang, G. Deng, J. D. Heath, N. Kurn, and P. Wangemann. 2005. Microarray-based comparison of three amplification methods for nanogram amounts of total RNA. Am J Physiol Cell Physiol 288:C1179-89.
116. Siragam, V., D. Brinc, A. R. Crow, S. Song, J. Freedman, and A. H. Lazarus. 2005. Can antibodies with specificity for soluble antigens mimic the therapeutic effects of intravenous IgG in the treatment of autoimmune disease? J Clin Invest 115:155-60.
117. Siragam, V., A. R. Crow, D. Brinc, S. Song, J. Freedman, and A. H. Lazarus. 2006. Intravenous immunoglobulin ameliorates ITP via activating Fc[gamma] receptors on dendritic cells. Nat Med 12:688-692.
118. Soundararajan, S., Y. Kikuchi, K. Joseph, and A. P. Kaplan. 2005. Functional assessment of pathogenic IgG subclasses in chronic autoimmune urticaria. Journal of Allergy and Clinical Immunology 115:815-821.
119. Stanberry, L. R. 1992. Pathogenesis of herpes simplex virus infection and animal models for its study. Curr Top Microbiol Immunol 179:15-30.
120. Tang, S. H., F. J. Silva, W. M. Tsark, and J. R. Mann. 2002. A Cre/loxP-deleter transgenic line in mouse strain 129S1/SvImJ. Genesis 32:199-202.
121. Thompson, K. A., W. W. Blessing, and S. L. Wesselingh. 2000. Herpes simplex replication and dissemination is not increased by corticosteroid treatment in a rat model of focal Herpes encephalitis. Journal of Neurovirology 6:25-32.
122. Toubi, E., and A. Etzioni. 2005. Intravenous immunoglobulin in immunodeficiency states: state of the art. Clin Rev Allergy Immunol 29:167-72.
123. UC-Davis September, 2001 2001, posting date. Herpes Simplex, full report. University of California at Davis May, 2003. [Online.]
124. Valyi-Nagy, T., S. L. Deshmane, B. Raengsakulrach, M. Nicosia, R. M. Gesser, M. Wysocka, A. Dillner, and N. W. Fraser. 1992. Herpes simplex virus type 1 mutant strain in1814 establishes a unique, slowly progressing infection in SCID mice. J Virol 66:7336-45.
125. van Lint, A., M. Ayers, A. G. Brooks, R. M. Coles, W. R. Heath, and F. R. Carbone. 2004. Herpes Simplex Virus-Specific CD8+ T Cells Can Clear Established Lytic Infections from Skin and Nerves and Can Partially Limit the Early Spread of Virus after Cutaneous Inoculation. J Immunol 172:392-397.
126. Wang, K., T. Y. Lau, M. Morales, E. K. Mont, and S. E. Straus. 2005. Laser-Capture Microdissection Refining Estimates of the Quantity and Distribution of Latent Herpes Simplex Virus 1 and Varicella-Zoster Virus DNA in Human Trigeminal Ganglia at the Single-Cell Level. J. Virol. 79:14079-14087.
127. Whitley, R. 2003. Herpsvirus Infections of the Central Nervous System. Herpes 11:1-84.
128. Whitley, R. J., D. W. Kimberlin, and B. Roizman. 1998. Herpes simplex viruses. Clin Infect Dis 26:541-53; quiz 554-5.
129. Whitley, R. J., and B. Roizman. 2001. Herpes simplex virus infections. Lancet 357:1513-8.
130. Wuhrer, M., J. C. Stam, F. E. van de Geijn, C. A. Koeleman, C. T. Verrips, R. J. Dolhain, C. H. Hokke, and A. M. Deelder. 2007. Glycosylation profiling of immunoglobulin G (IgG) subclasses from human serum. Proteomics 7:4070-81.
131. Yamamoto, M., Y. Hayashi, L. L. Tang, and R. Mori. 1985. Effects of combined use of acyclovir and antibody in athymic nude mice inoculated intracutaneously with herpes simplex virus. Antiviral Res 5:83-91.
132. Yang, L., C. C. Voytek, and T. P. Margolis. 2000. Immunohistochemical analysis of primary sensory neurons latently infected with herpes simplex virus type 1. J Virol 74:209-17.
133. Yoshimi, K., M. Woo, Y. Son, M. Baudry, and R. F. Thompson. 2002. IgG-immunostaining in the intact rabbit brain: variable but significant staining of hippocampal and cerebellar neurons with anti-IgG. Brain Research 956:53-66.

1. A method of treating a herpes simplex virus (HSV) infection in a mammal, the method comprising, administering to the mammal an effective amount of a composition comprising pooled immunoglobulin.

2. The method of claim 1 wherein the pooled immunoglobulin is prepared from human plasma or serum.

3. The method of claim 2, wherein the pooled immunoglobulin is intravenously injected.

4. The method of claim 2 wherein the mammal suffers from a herpetic disease or infection.

5. The method of claim 2, wherein the infection is a herpes simplex encephalitis or a herpes stromal keratitis.

6. The method of claim 5, wherein the encephalitis is associated with Japanese Encephalistis Virus, Venezuelan Equite Encephalitis Virus, or West Nile Virus.

7. The method of claim 1, wherein the immunoglobulin comprises sialylated IgG domains.

8. The method of claim 7, wherein the sialylated monomeric IgG domain is a monomeric Fc domain.

9. The method of claim 1, wherein the pooled immunoglobulin is parenterally administered.

10. A method of preventing a herpes simplex virus (HSV) infection in a mammal comprising intravenously administering to the mammal, prior to infection with HSV, an amount of a composition comprising pooled human immunoglobulin (IVIG) effective to prevent HSV infection.

11. The method of claim 10, wherein the immunoglobulin includes an Fc domain of sialylated monomeric IgG domain.

12. The method of claim 10, wherein the IVIG is administered in combination with at least one antiviral drug.

13. The method of claim 10, wherein the pooled immunoglobulin is processed for removal of one or more neutralizing antibody.

14. The method of claim 13, wherein the neutralizing antibody is a HSV neutralizing antibody.

15. The method of claim 13, wherein the immunoglobulin includes isolated Fc fragments enriched with sialic acid.

16. A composition comprising pooled human immunoglobulin, wherein the pooled human immunoglobulin comprises an Fc domain of sialylated monomeric IgG.

17. The composition of claim 16, wherein the pooled human immunoglobulin is prepared from human plasma or serum and comprises one or more HSV neutralizing antibodies.

18. The composition of claim 16, wherein the composition is administered to treat a mammal with HSV.

19. The composition of claim 18, wherein the mammal is a human and the HSV is herpes simplex encephalitis or a herpes stromal keratitis.
